(19) United States
(12) Patent Application Publication
(10) Pub. No.: US 2012/0020958 A1
(43) Pub. Date: Jan. 26, 2012

(54) FIBCD1 FOR THE PREVENTION AND TREATMENT OF DISEASES

(75) Inventors: Uffe Holmskov, Odense M (DK); Anders Schlosser, Odense M (DK)

(73) Assignee: Dansk Universitet, Odense M (DK)

(21) Appl. No.: 13/142,907

(22) PCT Filed: Dec. 30, 2008
(86) PCT No.: PCT/EP08/68362

§ 371 Date: Oct. 11, 2011

(51) Int. Cl. A61K 39/395; C08B 37/00; C07H 21/00; C08B 37/08; C07C 229/12; A61K 31/722; C07K 16/28; A61P 29/00; A61P 37/08; A61P 11/06; A61P 11/00; A61P 7/10; A61P 37/06; A61P 37/02; A61P 21/02; A61P 11/08; C07H 15/12

(52) U.S. Cl. 424/133.1; 536/53; 536/23.1; 536/20; 560/253; 424/184.1; 536/24.5; 530/389.8; 424/172.1

(57) ABSTRACT

The present invention relates to methods and compositions for the treatment and/or prevention of diseases such as allergic diseases and inflammatory bowel diseases comprising one or more FIBCD1 binding molecules or pharmaceutically acceptable salts, esters, and amides thereof and optionally one or more non-FIBCD1 binding molecules or pharmaceutically acceptable salts, esters, and amides thereof; as well as the use of such pharmaceutical composition for the prevention and/or treatment of diseases such as allergic diseases and inflammatory bowel diseases. FIBCD1 is herein defined as either a transmembrane receptor, the corresponding DNA or the corresponding mRNA as defined by SEQ ID No. 1, 2 and 3 as well as homologous thereof.

### TECHNICAL FIELD

This invention relates to methods and compositions for the treatment and/or prevention of diseases such as allergic diseases and inflammatory bowel diseases comprising one or more FIBCD1 binding molecules or pharmaceutically acceptable salts, esters, and amides thereof and optionally one or more non-FIBCD1 binding molecules or pharmaceutically acceptable salts, esters, and amides thereof.

### BACKGROUND

The cause of allergic diseases (or allergy) is the immune response to dietary or environmental substances known as allergens. Allergy is a form of hypersensitivity, more specifically called immediate (or type I) hypersensitivity. There are many substances which can cause allergic reactions; some of these substances are plant pollens, such as grass pollen and venom of stinging insects such as bees and wasps. Amongst the allergic responses are eczema, allergic rhinitis (hay fever), asthma, hives, food allergies and allergic reactions to the venom of stinging insects such as wasps and bees. Mild allergies like hay fever are widespread in the human population and cause symptoms such as itchiness, allergic conjunctivitis, and runny nose. Allergies can also play an important role in the development of conditions such as asthma. In some people, severe allergies to dietary-, environmental allergens, or to medication such as penicillin may result in life-threatening anaphylactic reactions which are potentially lethal. Since the occurrence of type I allergy in industrialized countries has increased immensely, it is now becoming a major health problem.

Allergy, as well as parasitic worm immunity, is characterized by infiltration of tissues with IL-4 and IL-13 expressing cells including T-helper-2 cells (Th2), eosinophiles and basophiles (Ramalingam T R et al., Curr Opin Allergy Clin Immunol. 2005, 5:392-398) and the tissue macrophages involved in the allergic response assume a distinct pheno-type designated alternative activated macrophages (Gordon S, Nat Rev immunol, 2003, 3:23-35). It has been hypothesized that the allergic response is a misdirected immune response against helminth infections. Chitin has recently been implicated as a pathogen-associated molecular pattern (PAMP) that modulates the allergic response (Reese T A, et al. Nature, 2007, 447:92-96).

Chitin was shown to induce an immune response characterized by infiltration of cells that express interleukin (IL)-4 and IL-13 including T-helper-2 cells, eosinophiles and basophiles (Reese, T A. et al., Nature, 2007, 447:92-96), a response that typically is seen in relation to an allergic and parasitic worm immune responses.

Chitin is a linear homopolymer of β-1,4-linked N-acetylglucosamine that next to cellulose is the most abundant known biopolymer (Kurita K., Mar Biotechnol (NY), 2006, 8:203-226). Chitin is an important structural component in the cell wall of most fungi (Latgé, J P et al., Mol Microbiol, 2007, 66:279-290), in the eggshell of parasitic nematodes (Govindan J A et al., Curr Biol, 2007, 17:R890-892), in the exoskeleton of all types of arthropods and in the cuticle of the epidermis and trachea and the lining of the gut of many insects (Merzendorfer, H. et al., J Exp Biol, 2003, 206:4393-4412). Vertebrates are therefore exposed to chitins through the ingested food when infected with nematodes or fungi.

Vertebrates lack the ability to produce chitin but in spite of this they do express highly conserved chitinases (Bussink, A P. et al., Genetics, 2007, 177:959-970). The acidic mammalian chitinase (AMCase) is expressed mainly by the salivary glands and by the stomach (Boot R G et al., J Biol. Chem., 2001, 276:6770-6778), while the chitotriosidase is expressed by tissue macrophages. Both are endo-β-1,4-N-acetylglucosaminidases that are believed to be involved in food digestion and immunity (Suzuki M, J Histochem Cytochem, 2002, 50:1081-1089). The human chitotriocidase was shown to have fungistatic effect (van Eijk, M., Int Immunol, 2005, 17:1505-1512) and the AMCase has been linked to the pathophysiology of asthma (Zhu, Z., Science, 2004, 304:1678-1682).

In plants, chitin and its fragments, chitin oligosaccharides or N-acetylchitooligosaccharides, are recognized as typical fungal PAMPs that triggers various defence responses. These include cell surface chitin recognition receptors like CEBiP (Kaku, H., Proc Natl Acad Sci USA, 2006, 103:11086-11089) and receptor-like kinases like CERK1 that elicits MARK activation, reactive oxygen species generation and gene expression upon activation with chitin (Miya, A. et al., Proc Natl Acad Sci USA, 2007, 104:19613-19618).

It has been shown that chitin instilled in the lung of mice (or given intraperitoneally) induced the accumulation of IL-4 and IL-13 expressing cells including Th2 cells, eosinophils and basophils (Reese, T A. et al., Nature, 2007, 447:92-96). This cell infiltration was inhibited by pre-treatment with AMCase or if the chitin was injected in mice overexpressing AMCase. The cell infiltration occurred independent of TLR4 or MyD88. Chitin mediated alternative macrophage activation in vivo and the production of leukotriene B₄, which was required for optimal immune cell recruitment.

Another group has shown, that oral administration of chitin down-regulates serum IgE levels and lung eosinophilia in an allergic mouse model (Shibata, Y., J Immunol, 2000, 164:1314-1321). This group used a mouse model, where chitin was given orally 3 days before and 13 days during ragweed allergen intraperitoneal immunization. The ragweed—immunized mice were then given ragweed intratracheally on day 11. Three days after, the immunized mice showed increased serum IgE levels and lung eosinophil numbers. The chitin treatment resulted in a decrease of these parameters. To understand the inhibitory mechanisms of Th2 responses, spleen cells isolated from the ragweed-immunized mice were cultured in the presence of ragweed and/or chitin for 3 days (recall responses). Ragweed alone stimulated the production of IL-4, IL-5, and IL-10, but not IFN-γ. Ragweed/chitin stimulation resulted in significant decreases of IL-4, II-5, and IL-10 levels and an increase in the production of IFN-γ production, suggesting that the immune responses were redirected toward a Th1 response.

These studies indicate, that chitin given orally during an allergic stimulus down-regulate Th2-facilitated IgE production and lung eosinophilia in the allergic mouse while chitin instillated directly in the lung have the opposite effect and that this effect can be inhibited by a chitinase.

In WO 04087874 is disclosed that a range of different polypeptides (approximately 470) may be used for treatment of inflammatory bowel disease and how the polypeptides (or antagonists thereof) may be used in the treatment of allergic reactions, including food allergies, insect venom allergies, atopic dermatitis, allergic conjunctivitis and allergic rhinitis. However, WO 04087874 does not disclose the specific use of a protein or polypeptide with the characteristics of FIBCD1, as disclosed herein, for the treatment og either inflammatory bowel diseases or allergic diseases.

In US 2008014281 A1 is disclosed how chitin micro-particles can be used for the therapeutically effective down regulation of Th2-mediated diseases such as allergic asthma, food allergy and allergic dermatitis. However, US 2008014281 A1 does not disclose that chitin binds to the FIBCD1 receptor or that other acetyl-containing compounds may be useful for the prevention or treatment of inflammatory bowel diseases or allergic diseases.

From the above it is clear that the increased number of people suffering from allergies necessitates the development of novel ways for the treatment of allergies. Thus, there is an urgent need for finding new molecular mechanisms which enable new ways of treatment or improve the effectiveness of current ways of treating allergic diseases.

Inflammatory bowel disease, which is a group of diseases that affect a large segment of the population, is believed to be caused by several different factors such as environment, diet, and possible genetics. Thus, there is also an urgent need for finding new molecular mechanisms which enable new ways of treatment or improve the effectiveness of current ways of treating inflammatory bowel diseases.

The object of the invention is to provide one or more FIBCD1 binding molecules or pharmaceutically acceptable salts, esters, and amides thereof which binds to—or otherwise modulates the effect of the naturally occurring FIBCD1 receptor; and optionally one or more non-FIBCD1 binding molecules or pharmaceutically acceptable salts, esters, and amides thereof, where said one or more non-FIBCD1 binding molecules binds to—or otherwise modulates the effect of a cellular target different from the naturally occurring FIBCD1 receptor, where said one or more FIBCD1 binding molecules or pharmaceutically acceptable salts, esters, and amides thereof and/or one or more non-FIBCD1 binding molecules or pharmaceutically acceptable salts, esters, and amides thereof is capable of preventing and/or treating diseases such as allergic diseases or inflammatory bowel diseases by administration of clinically relevant amounts of such one or more FIBCD1 binding molecules or pharmaceutically acceptable salts, esters, and amides thereof and/or one or more non-FIBCD1 binding molecules or pharmaceutically acceptable salts, esters, and amides thereof to a subject in need thereof.

### DISCLOSURE OF THE INVENTION

As will be understood from the above discussion, the prior art has not hitherto considered that compounds binding to—or otherwise modulating the effect of the naturally occurring FIBCD1 receptor are or could be involved in treatment and/or prevention of diseases such as allergic diseases or inflammatory bowel diseases.

Surprisingly, the present inventors have found that the naturally occurring membrane-bound receptor called FIBCD1 binds acetylated compounds, including chitin, which has been shown to be involved in the occurrence and development of allergies.

The present inventors have also shown that the naturally occurring membrane-bound FIBCD1 receptor is present in the small intestine. The microenvironment in the small intestine is in general involved in the antigen uptake and recognition by CD4+cells. The presentation of different antigens e.g. food proteins, commensal bacteria, fungi or invasive microorganisms may result in a different polarized immune response (Th1 versus Th2) resulting in either local/systemic tolerance or immunity (Mowat, A M. (2003) Nature reviews Immunology, 3, 331-341). The small intestine is also intimately involved in the development of inflammatory bowel diseases.

Accordingly, in a first aspect, the invention relates to the use of one or more FIBCD1 binding molecules which binds to—or otherwise modulates the effect of the naturally occurring FIBCD1 receptor and optionally one or more non-FIBCD1 binding molecules for the treatment and/or prevention of diseases such as allergic diseases and inflammatory bowel diseases.

Another aspect of the invention is pharmaceutical compositions for the treatment and/or prevention of diseases such as allergic diseases and inflammatory bowel diseases comprising one or more FIBCD1 binding molecules or pharmaceutically acceptable salts, esters, and amides thereof and optionally one or more non-FIBCD1 binding molecules or pharmaceutically acceptable salts, esters, and amides thereof.

A further aspect of the invention is methods for the treatment and/or prevention of diseases such as allergic diseases and inflammatory bowel diseases involving the use of one or more FIBCD1 binding molecules or pharmaceutically acceptable salts, esters, and amides thereof and optionally one or more non-FIBCD1 binding molecules or pharmaceutically acceptable salts, esters, and amides thereof.

Another aspect of the invention involves the use of one or more antibodies for the treatment and/or prevention of diseases such as allergic diseases and inflammatory bowel diseases, where said one or more antibodies inhibits or stimulates the function of FIBCD1.

Another aspect of the invention involves the use of one or more antisense oligonucleotides complementary to the mRNA encoding the naturally occurring FIBCD1 receptor for the treatment and/or prevention of diseases such as allergic diseases and inflammatory bowel diseases, where said one or more antibodies inhibits or stimulates the function of FIBCD1.

Other aspects of the invention will be apparent from the below description and the appended claims.

### BRIEF DESCRIPTION OF THE DRAWING(S)

The invention is explained in detail below with reference to the drawing(s), in which

FIG. 1 shows a schematic representation of FIBCD1. (A) SDS-PAGE and silver staining of purified FIBCD1-V5-His ectodomain in the reduced (1) and unreduced state (2). (B) Western blotting of FIBCD1-V5-His ectodomain after cross-linking with increasing concentration of the cross-linker BS-3 in the reduced state. (C) FIBCD1 ectodomain with and without enzymatic digest of N-linked glycans. (E) and (F) Type II membrane topology of FIBCD1 analysed by FACS and confocal microscopy analysis of CHO-FIBCD1/CHO cells using monoclonal mouse anti-FIBCD1 (HG-HYB-12-1).

FIG. 2 shows a sequence alignment of the fibrinogen like domains of FIBCD1, L-ficolin, M-ficolin, and tachylectin-5A (TL-5A). Numbers on top and below refer to L-ficolin and TL-5A sequences, respectively. The acetyl binding site residues are coloured green (S1), bold red (S2), black (S3) and bold orange (S4). Residues involved in Ca²⁺binding in L- and H-ficolin and TL-5A are shown in yellow (Garlatti et al 2007).

FIG. 3 shows that the ectodomain of FIBCD1 is an acetyl group-binding molecule. (A) SDS-PAGE and Western blotting analysis of culture supernatant from HEK293 cells expression FIBCD1 in the non-reduced (lane 1) and reduced (lane 2) state developed with the monoclonal rat anti-FIBCD1 (HG-HYB-12-2). (B) Culture supernatant from HEK293 cells expressing the ectodomain of FIBCD1 was run onto a column with NAc-TSK beats in TBS containing 5 mM CaCl₂ followed by wash in the same buffer and elution with buffer containing 250 mM sodium acetate. (D) Silver stained SDS-PAGE of fractions from (E) in the reduced and unreduced state. (E) Microtiterplates were coated with BSA or NAc-BSA and increasing amounts of FIBCD1 was added in the presence 5 mM CaCl₂ or 10 mM EDTA. (F) Microtiterplates were coated with NAc-BSA and 100 ng/ml and FIBCD1 was added in the presence 0-1 mM CaCl₂. (G) Microtiterplates were coated with NAc-BSA and increasing amounts of FIBCD1 was added in the presence 5 mM CaCl₂, 5 mM MgCl₂ or 5 mM MnCl₂. (H) Microtiterplates were coated with NAc-BSA and 100 ng/ml of FIBCD1 ectodomain was added in the presence of increasing concentrations of NAcMan, α-methyl-mannose, acetate or proprionate.

FIG. 4 shows that chitin specifically binds to FIBCD1 while other PAMPs like lipopolysaccharide (LPS), lipoteichoid acid (LTA), mannan, or peptidoglycan do not bind. (A) Microtiter plates were coated with LPS 055, LPS 0111 LPS 026 or NAc-BSA. (B) Microtiterplates were coated with LTA, mannan, peptidoglycan or NAc-BSA and increasing concentrations of FIBCD1 was added. (C) Pull-down assays where 20 μg of the FIBCD1 (the FReD of FIBCD1) or as a positive control 20 μg of wheat germ agglutinin (WGA) was added to 2 mg insoluble chitin, cellulose, zymozane or beta-glucan. (D) Pull-down assays where 20 μg of the FIBCD1 (the FReD of FIBCD1) was added to 2 mg insoluble chitin in the presence of calcium, calcium and 1 mM sodium acetate or EDTA. BSA was used as control. Pellet fractions were analyzed by SDS-PAGE and Coomassie blue staining.

FIG. 5 shows FIBCD1 mediated endocytosis of ¹²⁵I acetylated BSA. A, time course for cell asscociated (▪) and degraded (♦) ¹²⁵I acetylated BSA in CHO/CHOFIBCD1 cells. Confluent cell layers were incubated with ¹²⁵I acetylated BSA at 37° C. for various time intervals. Degradation was measured, as the cell-mediated increase in of TCA-soluble radioactivity in the growth medium and cell-associated was determined by counting the radioactivity of the cell lysate. (A) CHO cells and (B) CHO/FIBCD1 cells. (C) Effect of lysosomal inhibitors chloroquine and leupeptin (both 100 μM) on endocytosis of NAc-BSA. Values are mean+/−1 SD of triplicate samples. (D) Inhibition of ¹²⁵I acetylated BSA uptake. Fluorescence microscopy of CHO-FReD (E) and CHO (F) after incubation with Alexa488 acetylated BSA.

FIG. 6 shows the localization of FIBCD1 in human tissues. Formaldehyde fixed paraffin-embedded cells or tissues were incubated with the monoclonal antibody HG-HYB-12-2 directed against FIBCD1 and developed with biotin-labeled goat anti-mouse Ig, HRP-coupled streptavidin and substrate. Sections were counterstained with Mayer's haematoxylin. (A, B, and C) Duodenum; (D, E) colon; (F) salivary gland; (G, H) HEK293 with and without transfection FIBCD1 ectodomain cDNA. Original magnification: ×200 (A, D); ×400 (E, G, H) and ×600 (B, C, E).

FIG. 7 shows the chromosome localization, genomic organization and the coding sequence of FIBCD1, and the mRNA transcripts and deduced amino acid sequence of FIBCD1. The initiation methionine is marked as +1. The potential glycosylation site is marked by *. The transcription initiation site, the stop codon and polyadenylation site are boxed.

FIG. 8 shows alignment of the known FIBCD1 protein sequences using CRUSTAL V form the DNA Star package.

FIG. 9 shows real-time PCR analyses of FIBCD1 mRNA expression in human tissues. The real-time PCR analysis was correlated to uterus mRNA

FIG. 10 shows Western blot of triton-X100 lysates from HEK293 (lane 1) and HEK293 transfected with FIBCD1 ectodomain cDNA (lane 2) in the unreduced state (lanes 1 and 3) or reduced state (lanes 2 and 4). The western blot was developed using monoclonal anti-FIBCD1 antibody (HG-HYB-12-2).

FIG. 11 shows binding of different mutated forms of the FReD of FIBCD1 to acetylated BSA. Microtiter plates were coated with acetylated BSA or BSA and blocked with TBS/Tw before being incubated with FIBCD1 samples diluted in TBS/Tw (5 mM CaCl₂). The FIBCD1 samples were added from 1 μg/ml in a twofold dilution series. The signal from binding of protein to BSA was subtracted from the signal from binding to acetylated BSA. The resulting normalized signals were then expressed as a percentage of the signal from FIBCD1FReD at 1 μg/ml, which was set at 100%. The experiment was repeated three times.

### DETAILED DESCRIPTION OF THE INVENTION

In the present invention, it was found that the naturally occurring FIBCD1 receptor binds acetylated compounds and how the naturally occurring FIBCD1 receptor is involved in the development of diseases such as allergic diseases and inflammatory bowel diseases.

### DEFINITIONS

The term “naturally occurring” or any lingual variation thereof, refers to for example nucleic acid molecules or polypeptide molecules which are occurring or have occurred in nature or in natural systems.

The term “one or more” as used herein, refers to one, two, three, four, five, six, seven, eight, nine, ten, or more.

The term “homology” or any lingual variation thereof means that the respective nucleic acid molecules or encoded proteins are functionally and/or structurally equivalent. The nucleic acid molecules that are homologous to the nucleic acid molecules described herein and that are derivatives of said nucleic acid molecules are, for example, variations of said nucleic acid molecules which represent modifications having the same biological function, in particular encoding proteins with the same or substantially the same biological function. They may be naturally occurring variations, such as sequences from other animal varieties or species, or mutations. Structural equivalents can, for example, be identified by testing the binding of said polypeptide to antibodies or computer based predictions. Structural equivalents have the similar immunological characteristic, e.g. comprise similar epitopes.

The terms “prophylaxis”, “prophylactic treatment” or “prevention” refers to the treatment of a subject who does not yet have a disease or disorder, but who may be susceptible to, or at risk of getting a disease or disorder.

The term “subject” or any lingual variation thereof, means a living vertebrate animal, e.g., a mammal, such as a human being. The term also encompasses all animal disease models for e.g., asthma, allergy and inflammtory bowel disease and naturally occurring or non-naturally occurring mutated or non-human genetically engineered (e.g. transgenic or knockout) animals.

The term “treatment” or any lingual variation thereof, refers to a clinical endpoint characterized by an improvement in the subjects condition; a reduction in the severity, frequency, duration or progression of one or more adverse symptoms or complications associated with the disease or disorder; and/or an inhibition, reduction, elimination, prevention or reversal of one or more physiological, biochemical or cellular manifestations or characteristics of the disease or disorder, including complete prevention of the disease or disorder. Treatment as used herein also means the administration of an effective amount of a therapeutically active compound of the present invention with the purpose of easing, ameliorating, arresting, or eradicating (curing) symptoms or disease states.

The term “allergic diseases” or “allergy” or any lingual variation thereof, refers to a disorder of the immune system where dietary or environmental substances known as allergens cause a form of hypersensitivity, more specifically called immediate (or type I) hypersensitivity.

The term “inflammatory bowel diseases” or “IBD” or any lingual variation thereof, refers to a group of inflammatory conditions of the large- and small intestine. The major types of IBD are Crohn's disease and ulcerative colitis but also include collagenous colitis, lymphocytic colitis, ischaemic colitis, diversion colitis, Behcet's syndrome, infective colitis, indeterminate colitis, and/or other inflammatory bowel diseases.

The term fibrinogen C domain containing 1 “FIBCD1” include any variants, isoforms and species homologues of human FIBCD1, which are naturally expressed by cells or are expressed on cells transfected with the FIBCD1 gene, see also: http://www.genecards.org/cgi-bin/carddisp.pl?gene=FIBCD1. Synonyms (aliases) of FIBCD1, as recognized in the art, FLJ14810 (HGNC, Entrez Gene): OTTHUMP00000022378 2 (HGNC). Human FIBCD1 has external Ids: HGNC: 259221; Entrez Gene: 849292; UniProt: □8N5393; Ensembl: ENSG000001307207.

FIBCD1 as defined herein by SEQ ID No. 1, may also comprise homologues which are at least 95% homologous with SEQ ID No. 1, such as at least 96% homologous, at least 97% homologous, at least 98% homologous, or at least 99% homologous. The cDNA sequence encoding for the FIBCD1 receptor is shown in SEQ ID No. 2 and may also comprise homologues which are at least 95% homologous with SEQ ID No. 2, such as at least 96% homologous, at least 97% homologous, at least 98% homologous, or at least 99% homologous. The mRNA sequence encoding for the FIBCD1 receptor is shown in SEQ ID No. 3 and may also comprise homologues which are at least 95% homologous with SEQ ID No. 3, such as at least 96% homologous, at least 97% homologous, at least 98% homologous, or at least 99% homologous.

The term “FIBCD1 binding molecules” or any lingual variation thereof, refers to any molecule or chemical entity that specifically binds to at least a portion of FIBCD1 under cellular and/or physiological conditions for an amount of time sufficient to inhibit the activity of FIBCD1 expressing cells and/or otherwise modulate a physiological effect associated with FIBCD1; to allow detection by ELISA, western blot, or other similarly suitable binding techniques described herein and/or known in the art and/or to otherwise be detectably bound thereto after a relevant period of time (for instance at least about 15 minutes, such as at least about 30 minutes, at least about 45 minutes, at least about 1 hour, at least about 2 hours, at least about 4 hours, at least about 6 hours, at least about 12 hours, such as about 1-24 hours, about 1-36 hours, about 1-48 hours, about 1-72 hours, about one week, or longer).

The term “partially deacetylated” or any lingual variation thereof, refers to any of the herein mentioned FIBCD1 binding molecules which contain a number of acetyl groups which is less than the maximum number of acetyl groups possible. As a non limiting example, chitin may be partially deactetylated, which essentially means that the chitin oligomer or polymer comprises one or more monomer units of glucosamine as well as one or more monomer units of N-acetylglucosamine. “Partially deacetylated” can be defined as a degree of acetylation less than 100%, where the degree of acetylation is determined as n_{Ac}/(n_{Ac}+n_{non-Ac}),

where n_{Ac} is the total number of acetyl-bearing groups and n_{non-Ac} is the total number of possible acetyl-bearing groups which are deacetylated. The FIBCD1 binding molecules defined herein may be about 1-10% deacetylated, 10-20% deacetylated, 20-30% deacetylated, 30-40% deacetylated, 40-50% deacetylated, 50-60% deacetylated, 60-70% deacetylated, 70-80% deacetylated, 80-90% deacetylated, 90-95% deacetylated, or 95-99% deacetylated. It is particular desirable to obtain a degree of deacetylation which affords FIBCD1 binding molecules which are soluble under physiological conditions.

The term “non-FIBCD1 binding molecules” or any lingual variation thereof, refers to a compound or chemical entity which binds to—or otherwise modulates the effect of a cellular target different from the naturally occurring FIBCD1 receptor. Examples of such a “non-FIBCD1 binding molecule” are numerous and may comprise: anti-asthma drugs, anti-allergy drugs, anti-histamine drugs, smooth muscle cell relaxing drugs, mast-cell stabilizers, anti-IgE drugs, selective or non-selective potassium channel activators (bronchodilators), immunomodulating agents, mucus secretion inhibitors, mucus liquefying agents, leukotriens modifier drugs, leukotriens receptor antagonists, mesalamine, steroids, prednisone, Asacol, and Immunosuppressants such as prednisone, infliximab (Remicade), azathioprine (Imuran), methotrexate, or 6-mercaptopurine.

“Pharmaceutically acceptable salts, esters, and amides” include carboxylate salts (e.g. C₁₋₈ alkyl, cycloalkyl, aryl, heteroaryl, or non-aromatic heterocyclic) amino acid addition salts, esters, and amides which are within a reasonable benefit/risk ratio, pharmacologically effective and suitable for contact with the tissues of patients without undue toxicity, irritation, or allergic response. Representative salts include hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, and laurylsulfonate. These may include alkali metal and alkali earth cations such as sodium, potassium, calcium, and magnesium, as well as non-toxic ammonium, quaternary ammonium, and amine cations such as tetramethyl ammonium, methylamine, trimethylamine, and ethylamine. See example, S. M. Berge, et al., “Pharmaceutical Salts,”J. Pharm. Sci., 1977, 66: 1-19 which is incorporated herein by reference. Representative pharmaceutical acceptable amides of the invention include those derived from ammonia, primary C₁₋₆ alkyl amines and secondary di(C₁₋₆ alkyl) amines. Secondary amines include 5- or 6-membered heterocyclic or heteroaromatic ring moieties containing one or more nitrogen atoms and optionally between 1 and 2 additional heteroatoms.

Preferred amides are derived from ammonia, C₁₋₃ alkyl primary amines, and di(C₁₋₂ alkyl) amines. Representative pharmaceutical acceptable esters of the invention include C₁₋₇ alkyl, C₅₋₇ cycloalkyl, phenyl, and phenyl(C₁₋₆) alkyl esters. Preferred esters include alkyl esters such as methyl esters, ethyl ester or propyl esters.

The term “immunoglobulin” or any lingual variation thereof, refers to a class of structurally related glycoproteins consisting of two pairs of polypeptide chains, one pair of light (L) low molecular weight chains and one pair of heavy (H) chains, all four interconnected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H}) and a heavy chain constant region. The heavy chain constant region, C_{H}, typically is comprised of three domains, C_{H}1, C_{H}2, and C_{H}3. Each light chain typically is comprised of a light chain variable region (abbreviated herein as V_{L}) and a light chain constant region. The light chain constant region typically is comprised of one domain, C_{L}. The V_{H} and V_{L} regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each V_{H} and V_{L} is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). Typically, the numbering of amino acid residues in this region is performed by the method described in Kabat et al., Sequences of Proteins of Immunological Interest, 5th Ed. Public Health Service, National Institutes of Health, Bethesda, Md. (1991) (phrases, such as variable domain residue numbering as in Kabat or according to Kabat herein refer to this numbering system for heavy chain variable domains or light chain variable domains). Using this numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (for instance residue 52a according to Kabat) after residue 52 of V_{H} CDR2 and inserted residues (for instance residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The term “antibody” or any lingual variation thereof, refers to an immunoglobulin molecule, a fragment of an immunoglobulin molecule, or a derivative of either thereof, which has the ability to specifically bind to an antigen under typical physiological conditions for a significant period of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or a time sufficient for the antibody to recruit an Fc-mediated effector activity). The variable regions of the heavy and light chains of the immunoglobulin molecule contain a binding domain that interacts with an antigen. The constant regions of the antibodies may mediate the binding of the immunoglobulin to host tissues or factors, including various cells of the immune system (such as effector cells) and components of the complement system such as C1q, the first component in the classical pathway of complement activation.

The anti-FIBCD1 antibody may be mono-, bi- or multispecific. Indeed, bispecific antibodies, diabodies, and the like, provided by the present invention may bind any suitable target in addition to a portion of FIBCD1. As indicated above, the term “antibody” as used herein, unless otherwise stated or clearly contradicted by the context, includes fragments of an antibody provided by any known technique, such as enzymatic cleavage, peptide synthesis and recombinant techniques that retain the ability to specifically bind to an antigen. It has been shown that the antigen-binding function of an antibody may be performed by fragments of a full-length (intact) antibody. Examples of antigen-binding fragments encompassed within the term “antibody” include, but are not limited to (i) a Fab fragment, a monovalent fragment consisting of the V_{L}, V_{H}, C_{L} and C_{H}1 domains; (ii) F(ab)₂ and F(ab′)₂ fragments, bivalent fragments comprising two Fab fragments linked by a disulfide bridge at the hinge region; (iii) a Fd fragment consisting essentially of the V_{H} and C_{H}1 domains; (iv) a Fv fragment consisting essentially of the V_{L} and V_{H} domains of a single arm of an antibody, (v) a dAb fragment (Ward et al., Nature 341, 544-546 (1989)), which consists essentially of a V_{H} domain and also called domain antibodies (Holt et al. (November 2003) Trends Biotechnol. 21(11):484-90); (vi) a camelid antibody or nanobody (Revets et al. (January 2005) Expert Opin Biol Ther. 5(1):111-24), (vii) an isolated complementarity determining region (CDR), such as a V_{H} CDR3, (viii) a UniBody™, a monovalent antibody as disclosed in WO 2007/059782, (ix) a single chain antibody or single chain Fv (scFv), see for instance Bird et al., Science 242, 423-426 (1988) and Huston et al., PNAS USA 85, 5879-5883 (1988)), (x) a diabody (a scFv dimer), which can be monospecific or bispecific (see for instance PNAS USA 90(14), 6444-6448 (1993), EP 404097 or WO 93/11161 for a description of diabodies), a triabody or a tetrabody.

It should be understood that the term antibody generally includes monoclonal antibodies, polyclonal antibodies as well as fragments thereof. The antibodies can be human, humanized, chimeric, murine, etc.

As used herein, “specific binding” or “specifically binds to” or any lingual variations thereof, refers to the binding of a binding molecule, such as an acetylated compound, an antisense oligonucleotide, a full-length antibody, or an antigen-binding fragment thereof, to a predetermined antigen. Typically, the antibody binds with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less, when measured for instance using sulfon plasmon resonance on BIAcore or as apparent affinities based on IC₅₀ values in FACS or ELISA, and binds to the predetermined antigen with an affinity corresponding to a K_{D} that is at least ten-fold lower, such as at least 100 fold lower, for instance at least 1000 fold lower, such as at least 10,000 fold lower, for instance at least 100,000 fold lower than its affinity for binding to a non-specific antigen (e.g., BSA, casein) other than the predetermined antigen or a closely-related antigen. When the K_{D} of the antigen binding peptide is very low (that is, the antigen binding peptide is highly specific), then the affinity for the antigen may be at least 10,000 or 100,000 fold lower than the affinity for a nonspecific antigen.

The terms “antisense strand”, “antisense oligonucleotide” or any lingual variations thereof, refers to a single stranded poly- or oligonucleotide which has an “anti sense” base sequence which is complementary to the mRNA, having the sense base sequence, which it targets. However, herein an “antisense strand” or “antisense oligonucleotide” also refers to a single stranded poly- or oligonucleotide which has an “anti sense” base sequence which is complementary to the coding DNA strand, having the sense base sequence, which it targets—even though, conventionally, this is strictly called “antigene” since it targets DNA instead of mRNA. The antisense oligonucleotide may comprise both natural and/or unnatural nucleotides, such as DNA-, RNA-, PNA- and/or LNA nucleotides.

The term “allergen” or any lingual variation thereof, refers to all foreign agents or substances capable of inducing, promoting, or stimulating allergy, i.e. the hypersensitive state induced by an exaggerated immune response to the allergen, or asthmatic reaction in a subject. The term “allergen” includes weed/plant/tree pollens or spores, animal dander, house dust mite, dust, lint, mite feces, fungal spores, wasp-venom, bee-venom, fire ant-venom, penicillin, sulfonamides, food ingredients, latex and cockroaches. The most common allergens, which cause allergic reactions, include inhalation allergens originating from trees, herbs, weed, plants, grasses, fungi, house dust mites, storage mites, cockroaches and animal hair, feathers, and dandruff. Important pollen allergens from trees, grasses, weeds and herbs are such originating from the taxonomic orders of Fagales, Oleales and Pinales including birch (Betula), alder (Alnus), hazel (Corylus), hornbeam (Carpinus) and olive (Olea), the order of Poales including grasses of the genera Lolium, Phleum, Poa, Cynodon, Dactylis and Secale, the orders of Asterales and Urticales including i. a. herbs of the genera Ambrosia and Artemisia. Important inhalation allergens from fungi are i.a. such originating from the genera Alternaria and Cladosporium. Other important inhalation allergens are those from house dust mites of the genus Dermatophagoides, storage mites from the genus Lepidoglyphys destructor, those from cockroaches and those from vertebrate such as cat, dog, horse, cow, and bird. Also, allergic reactions towards stinging or biting insects such as those from the taxonomic order of Hymenoptera including bees, wasps, and ants are commonly observed. Specific allergen components are known to the person skilled in the art and include e.g. Bet v 1 (B. verrucosa, birch), Aln g 1 (Alnus glutinosa, alder), Cor a 1 (Corylus avelana, hazel) and Car b 1 (Carpinus betulus, hornbeam) of the Fagales order. Others are Cry j 1 (Pinales), Amb a 1 and 2, Art v 1 (Asterales), Par j 1 (Urticales), Ole e 1 (Oleales), Ave e 1, Cyn d 1, Dac g 1, Fes p 1, Hol 11, Lol p 1 and 5, Pas n 1, Phl p 1 and 5, Poa p 1, 2 and 5, Sec c 1 and 5, and Sor h 1 (various grasspollens), Alt a 1 and Cla h 1 (fungi), Der f 1 and 2, Der p 1 and 2 (house dust mites, D. farinae and D. pteronyssinus, respectively), Lep d 1, Bla g 1 and 2, Per a 1 (cockroaches, Blatella germanica and Periplaneta americana, respectively), Fel d 1 (cat), Can f 1 (dog), Equ c 1, 2 and 3 (horse), Apis m 1 and 2 (honeybee), Ves g 1, 2 and 5, Pol a 1, 2 and 5 (all wasps) and Sol i 1, 2, 3 and 4 (fire ant), to mention the most common.

Non-limiting examples of weed, plant or tree pollen include:
weed pollen such as, but not limited to, dandelion, goldenrod, nettle, sage, clover, ragweed, mugwort, pellitory, nettles and dock;
grass pollen such as, but not limited to, Bermuda couch grass, sweet vernal grass, red and blue grasses, Johnson grass pollen; ryegrass such as Italian or annual rye-grass, perennial ryegrass, hybrid ryegrass, timothy grass, orchard grass, tall fescue, meadow fescue and red fescue;
tree pollen such as, but not limited to, alder, oak, ash, cypress, olive, maple, cedar, western red cedar, elm, birch, hickory, poplar, American sycamore, and walnut; and
annual plant pollen such as, but not limited to, tobacco and cotton.

Animal allergens may for example be skin, hair, various parasites and fungi.

Food allergens may for example comprise proteins from milk, milk products, eggs, peanuts, tree nuts, seafood, shellfish, rice, celery, buckwheat flour, soba noodles, red meat, soy or wheat.

Examples of a “disease” or a “disorder” or any lingual variation thereof include, but are not limited to, allergic asthma, asthma, extrinsic bronchial asthma, chronic obstructive pulmonary disease, hay fever (seasonal allergic rhinitis), allergic rhinitis, allergic conjunctivitis, hives, eczema, urticaria, angioedema, onchocercal dermatitis, atopic dermatitis, dermatitis, swelling, hypersensitivity pneumonitis, bronchopulmonary dysplasia, food allergy, Crohn's disease, ulcerative colitis, collagenous colitis, lymphocytic colitis, ischaemic colitis, diversion colitis, Behcet's syndrome, infective colitis, indeterminate colitis, and other inflammatory bowel diseases or other allergic diseases.

The term “asthma” or any lingual variation thereof refers to an allergic or non-allergic condition, disorder or disease of the respiratory system that is episodic and characterized by inflammation with constriction, narrowing or obstruction of the airways. Allergic asthma is typically associated with increased reactivity of the respiratory system to an inhaled allergen. Asthma is often, although not exclusively associated with atopic or allergic symptoms. Typically, a subject with asthma suffers from recurrent attacks of cough, shortness of breath with wheezing, chest pain, chest tightness, etc. Asthmatic conditions can be acute, chronic, mild, moderate or severe asthma (unstable asthma), nocturnal asthma or asthma associated with psychological stress.

The term “allergic rhinitis” or any lingual variation thereof refers to an allergic reaction of the nasal mucosa (upper airways), which includes hay fever (seasonal allergic rhinitis) and perennial rhinitis (non-seasonal allergic rhinitis) which are typically characterized by seasonal or perennial sneezing, rhinorrhea, nasal congestion, pruritis and eye itching, redness and tearing.

### The FIBCD1 Protein

As already mentioned, the invention is based on targeting FIBCD1 at the DNA, mRNA and/or protein level to treat or prevent diseases such as allergic diseases and inflammatory bowel diseases. The amino acid sequence of the FIBCD1 receptor molecule is shown in SEQ ID No. 1, the cDNA sequence encoding for the FIBCD1 receptor is shown in SEQ ID No. 2, and the mRNA sequence encoding for the FIBCD1 receptor is shown in SEQ ID No. 3.

The ability of the FIBCD1 molecule to bind to molecules like chitin and the linkage to chitin being involved in allergic responses is the rationale for the present invention. FIBCD1 is unlike other receptors with characteristics similar to FIBCD1—among other places located in the epithelium membrane of the small intestine. Thus, due to the localisation of FIBCD1 in the small intestine, the present inventors have also found that FIBCD1 may be involved in the development of inflammatory bowel diseases. Highly conserved homologues of FIBCD1 are found in rat and mouse. A recombinant form of the extracellular part of FIBCD1 expressed in 293 cells in a mammalian expression system forms a disulfide linked homotetramer of 250 kDa polypeptides. Three conserved cysteines found in the proximal extracellular part of the membrane may facilitate this oligomerization. One potential N-linked glycosylation site is found in the Fibrinogen-Related Domain (FReD). Real-time PCR on 22 different human tissues showed the highest levels of FIBCD1 expression in testis, adrenal gland, brain, mammary gland, retina, placenta, colon and lung.

Structure based alignment suggests that FIBCD1, like L-ficolin (Krarup, A et al., J. Biol. Chem. 279; 47513 (2004)), M-ficolin (Frederiksen, P. D. et al., Scand. J. Immunol. 62; 462 (2005)) and tachylectin 5's (Gokudan, S. et al., Proc. Natl. Acad. Sci. USA 96; 10086 (1999)), may recognize acetyl group containing substances, including acetylated carbohydrates. The acetyl group has now been shown to be sufficient for the recognition of these proteins.

The region between the coiled coil and the FReD domain was found to be highly cationic with a very high density of arginine residues. This cationic region is similar to cationic regions found in scavenger receptors like SR-A, CD36 and CL-P1, where this region binds polyanionic ligands as oxLDL, bacterial products, extracellular matrix, apoptotic cells and polyanionic polysaccharide (Platt, N. and Gordon, S., J. Clin. Invest. 108; 649 (2001); Ohtani, K. et al., J. Biol. Chem. 276; 44222 (2001); Kunjathoor, V. V. et al., J. Biol. Chem. 277; 49982 (2002)).

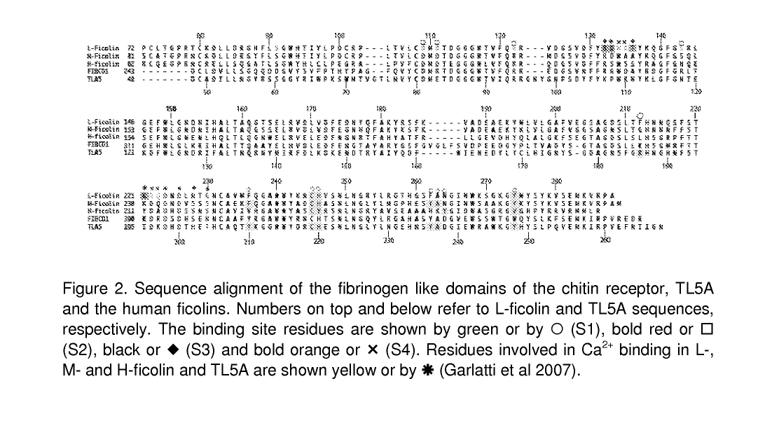

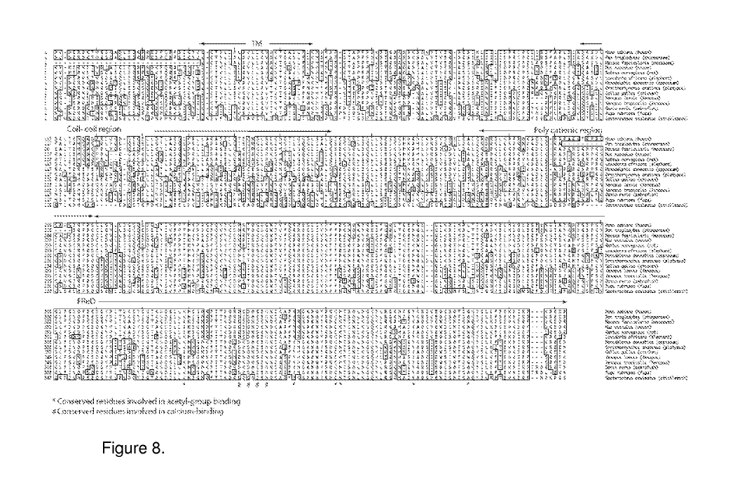

The present inventors have now shown that the FReD of FIBCD1 shows homology to the acetyl group binding innate immunity proteins L-ficolin, M-ficolin, tachylectin5A (TL-5A), and have found that FIBCD1 may have a similar role (FIG. 2). A distinct difference is however that the FIBCD1 sequence predicts a membrane protein. Further investigation revealed that FIBCD1 is highly conserved in vertebrates from mammals to birds, amphibians and fish (FIG. 8). Insects and worms also express membrane bound FReDs, but in these molecules the conserved residues involved in acetyl-group binding are lost.

### Structural Characterization of the FIBCD1 Ectodomain

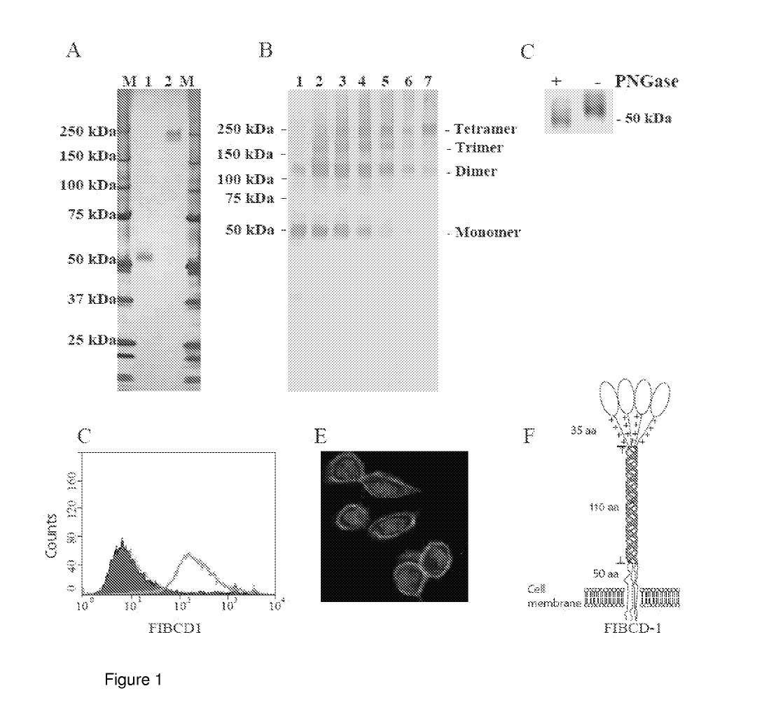

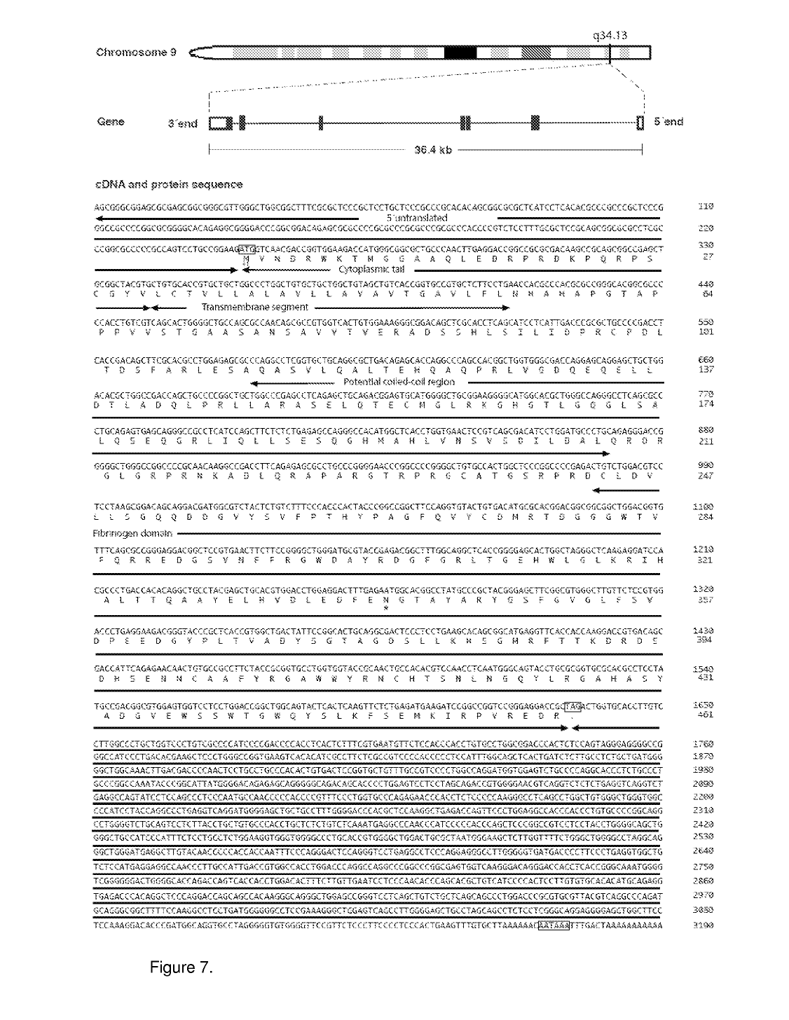

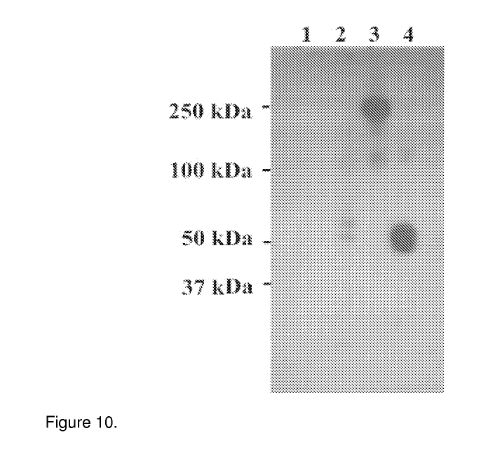

The present inventors expressed the ectodomain of FIBCD1 as a secreted protein in the human cell line HEK293 to get insight into the structural organization of FIBCD1. The purified protein migrates corresponding to a molecular mass of approximately 55 kDa in the reduced state and 250 kDa in the unreduced state on SDS-PAGE (FIG. 1A). This demonstrates that FIBCD1 is assembled as a disulfide linked homopolymeric structure. One cysteine is located in the region of potential coiled-coils and two cysteines are located at the boundaries (FIG. 7) and all three cysteines can potentially be involved in the interchain disulfide bridge formation. The present inventors have performed chemical cross-linking of the FIBCD1 ectodomain, which resulted in four distinct bands corresponding to monomeric, dimeric, trimeric and tetrameric structures, when analysed in the reduced state by SDS-PAGE (FIG. 1B). Treatment with N-glycanase reduced the molecular mass of FIBCD1 with 2-3 kDa (FIG. 10). The present inventors have, by FACS analysis (FIG. 1D) and immunofluorescence confocal microscopy analysis (FIG. 1E) of CHO cells expressing FIBCD1, shown that FIBCD1 is a membrane protein with type II topology. Together this indicates that FIBCD1 is a glycoprotein, which forms disulfide-linked homotetramers in the plasma membrane. A model of FIBCD1 is shown in FIG. 1F. Pattern recognition receptors often form oligomers. The increased number of binding sites increases the affinity for ligands and it helps to discriminate pathogen-associated molecular patterns (PAMPs) from potential self-ligands that are not ordered in distinct patterns.

### FIBCD1 is a Calcium-Dependent Acetyl-Group Binding Molecule

The FReD of FIBCD1 displays homology to the FReDs of the TL5A and to L- and M-ficolin. These proteins are known to bind acetylated components and they have been purified using acetyl group based affinity chromatography. The alignment of the human ficolins with TL5A and FIBCD1 reveals that some but not all of the residues of the TL5A and L- and M-ficolin involved in the NAc binding site are conserved in FIBCD1 (FIG. 2).

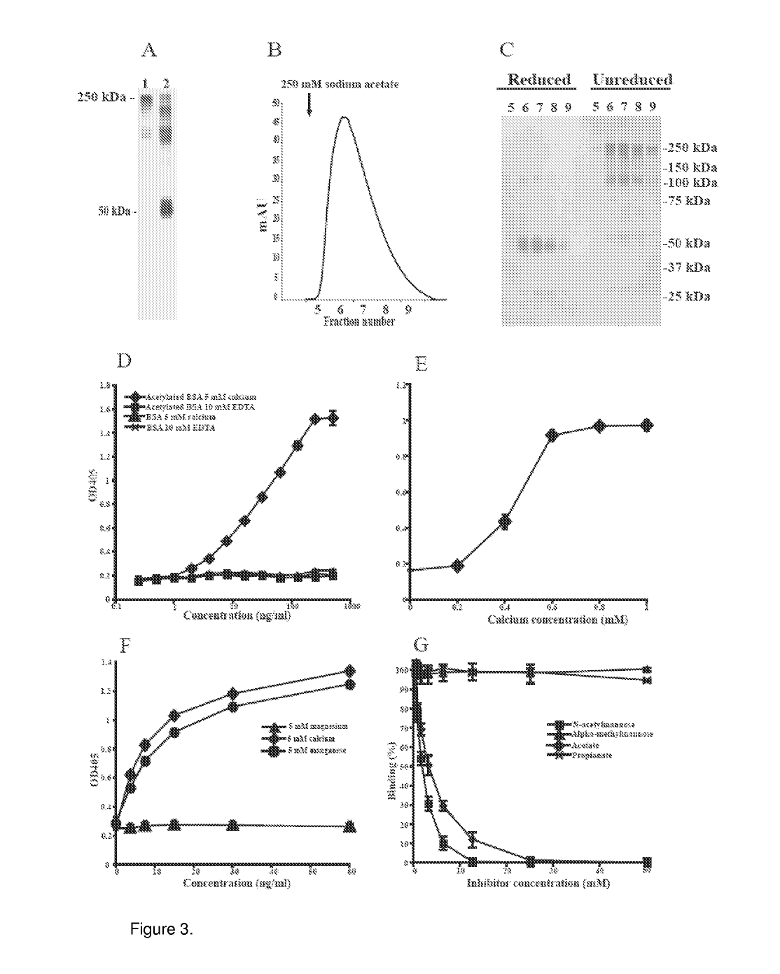

The present inventors have purified the FIBCD1 ectodomain by affinity chromatography on acetylated Toyopearls (Gokudan, S. et al., Proc. Natl. Acad. Sci. USA 96; 10086 (1999). FIG. 3A shows a Western blot of the culture supernatant from HEK293 cells expressing FIBCD1. FIG. 3B shows the acetate elution profile from the acetylated Toyopearls and silver stained SDS-PAGE of the purified FIBCD1 ectodomain is shown in FIG. 3C. This shows that FIBCD1—like ficolin L, M and TL5A—can be affinity purified using an acetate-coupled matrix.

The present inventors then further tested the ability of FIBCD1 to bind acetylated molecules by an ELISA based approach. The purified FIBCD1 ectodomain bound acetylated BSA coated microtiter plates in the presence of calcium. The binding was saturable and abolished by chelating of divalent ions (FIG. 3D). The binding between FIBCD1 and acetylated BSA was tested in the presence of increasing concentrations of calcium chloride (FIG. 3E) and CaCl₂ was substituted by other divalent cations (FIG. 3F). These experiments show that optimum binding to acetylated BSA is achieved at a calcium concentration of 0.6 mM and that manganese could substitute for calcium whereas magnesium could not.

The ligand selectivity of FIBCD1 was investigated by inhibiting the binding of FIBCD1 ectodomain to acetylated BSA coated microtiter plates (FIG. 2G, Table 1). The inhibition experiments demonstrated that N-acetylated carbohydrates or amino acids, but not their corresponding non-acetylated counterparts could inhibit the binding. Furthermore, one of the simplest compounds containing the acetyl group, sodium acetate, strongly inhibited the binding even at millimolar levels whereas sodium propionate and sodium butyrate did not inhibit at a concentration of 50 mM. Other acetylated compounds like acetylcholine could also inhibit the binding of FIBCD1 to acetylated BSA (Table 1).

TABLE 1
I₅₀ values of the binding between FIBCD1 and acetylated BSA. I₅₀
calculated from inhibition curves as shown in FIG. 1 B). The relative
inhibitor potential (“relative”) was determined by dividing the I₅₀ of
the best inhibitor with the I₅₀ of the desired compound. N.I., not
inhibitory (i.e. resulting in less than 50% inhibition at 50 mM).

| Inhibitor | I₅₀ (mM) | I₅₀ relative |
|---|---|---|
| Sialic acid | 2 | 1 |
| Glucose | N.I. | |
| Glucosamine | N.I. | |
| N-acetylglucosamine | 4 | 0.5 |
| Mannose | N.I. | |
| Mannosamine | N.I. | |
| N-acetylmannoseamine | 2 | 1 |
| Galactose | N.I. | |
| Galactoseamin | N.I. | |
| N-acetylgalactoseamine | 5 | 0.4 |
| Alanine | N.I. | |
| N-acetylalanine | 4 | 0.5 |
| Acetylcholine | 10 | 0.2 |
| Acetate | 3 | 0.7 |
| Butyrate | N.I. | |
| Propionate | N.I. | |

The acetyl-group specificity of TL5A was explained by the crystal structure of TL5A in complex with GlcNAc (Kairies N et al (2001) Proc Natl Acad Sci USA. 98:13519-24). Four aromatic site chains, Tyr-210, Tyr-236, Tyr-248 and His-220 form a funnel, with the methyl side chain of Ala-237 at its base. The methyl group of the acetylated carbohydrate benefits from the hydrophobic environment and lies in the middle of the funnel, surrounded by the aromatic site chains and in close van der Walls contact with the site chain methyl group of Ala-237. An unusual cis-peptide bond between Arg-218 and Cys-219 generates a sharp turn and allows the backbone NH group of the Cys to form a tight hydrogen bond with the carbonyl oxygen of the ligand acetamido group. The same cis-peptide bond was found in L-ficolin between Ans244 and Cys245 (Garlatti V et al (2007) EMBO J. 26:623-33), and in M-ficolin between Asp253-Cys254 (Garlatti V (2007) J Biol. Chem. 282:35814-20) and the present inventors speculate that the same can be the case for the homologous Asn413 and Cys414 residues in FIBCD1.

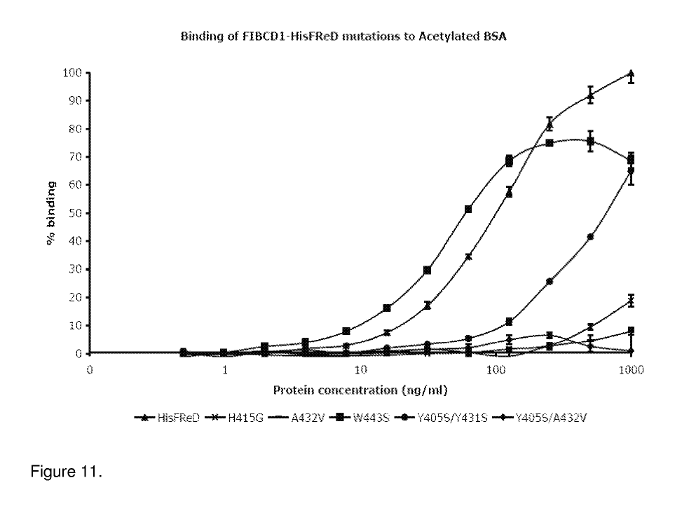

The hydrophobic funnel containing the methyl group of the acetylated carbohydrate is essentially conserved in FIBCD1 the Tyr248 being substituted with a Trp (FIG. 2). The homologue binding site (FIG. 2, S1) is also present in L- and M-ficolin. In M-ficolin the binding site is occupied by acetylated carbohydrates, but although L-ficolin binds acetylated compounds with high affinity, no ligands were found in the hydrophobic funnel when analyzing the crystal structure of L-ficolin. The present inventors have performed site directed mutagenesis on different amino acid residues potentially involved in FIBCD1 acetyl-group binding. Two single mutations (H415G and A432V) completely arrogate the binding of FIBCD1 to NAc-BSA and W443S, while W443S partially arrogated the NAc-BSA binding. Further structural analysis are needed to get a complete picture of the acetyl-group binding of FIBCD1 (FIG. 11).

The structure of L-ficolin revealed three additional binding sites (FIG. 2, S2-S4) and most of the acetylated ligands were found to bind to site (FIG. 2, S3) (Garlatti V et al (2007) EMBO J. 26:623-33), The acetyl moiety of these ligands bound to the backbone nitrogen of L-ficolin Asp133, a residue not conserved in either TL5A or FIBCD1. Galactose bound to (FIG. 2, S2) and the first three of four glucose residues of β-1,3-glucan were spanning the binding sites (FIGS. 2, S3 and S4). These binding sites were not conserved in FIBCD1.

### FIBCD1 Binds to Chitin

Chitin is a linear homopolymer of β-1,4-linked N-acetylglucosamine that next to cellulose is the most abundant known biopolymer (Kurita, K. 2006. Mar Biotechnol (NY) 8:203-226). Chitin is an important structural component in the cell wall of most fungi (Latge, J. P. 2007. Mol Microbiol 66:279-290.) in the eggshell of nematodes (Govindan, J. A., and D. Greenstein. 2007 Curr Biol 17:R890-892), in the exoskeleton of all types of arthropods and in the cuticle of the epidermis and trachea and the lining of the gut of many insects (Merzendorfer, H., and L. Zimoch. 2003 J Exp Biol 206:4393-4412). Vertebrates are therefore exposed to chitins both through the ingested food and when infected with nematodes or fungi.

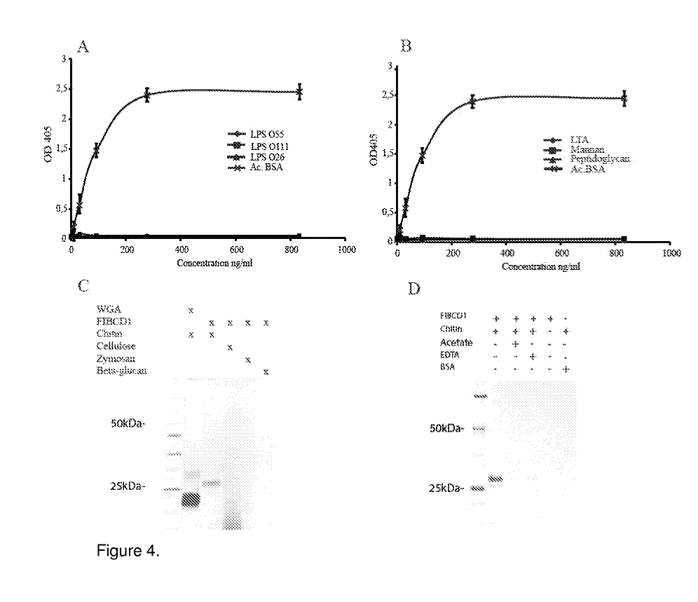

The present inventors have now shown that the FIBCD1 receptor specifically targets acetyl-containing compounds, such as chitin. The inventors have analysed the binding of chitin to FIBCD1 along with mannan, zymosane, LPS, peptidoglycan, LTA and β-1,3-glucan, the last three being known ligands for L-ficolin. As shown in FIG. 4A binding to the NAc-BSA was observed, when analysed by ELISA, while no binding was observed to LPS (FIG. 4A), mannan, peptidoglycan or to LTA (FIG. 4B). The FIBCD1 chitin binding activity was further detected in pull-down assays. As for the binding to NAc-BSA the binding was calcium dependent and could be inhibited by sodium acetate (FIG. 4D). Parallel pull-down experiments using equivalent masses of zymosan, cellulose the non-acetylated counterpart to chitin or β-1,3-glucan showed no binding. This implies that FIBCD1, in spite of the similarity between the carbohydrate backbone of chitin and peptidoglycan, has specific preference for chitin.

Uptake of ¹²⁵I-Labeled ac-BSA in FIBCD1-Expressinq CHO Cells

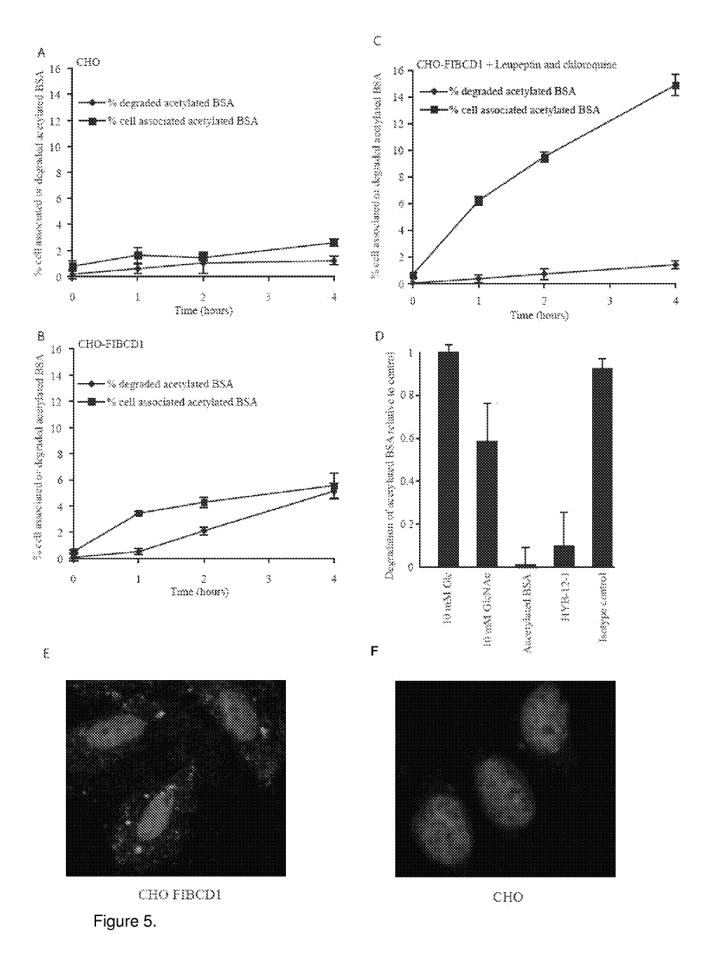

FIBCD1-mediated endocytosis of ¹²⁵I-labeled NAc-BSA was studied in Chinese hamster ovary (CHO) cells transfected with FIBCD1 cDNA. FIG. 5 shows the time course of cell-associated radioactivity and trichloroacetic acid (TCA)-soluble radioactivity (representing degraded ligand) in the medium (FIG. 5 A, B). The cell-associated radioactivity reached a plateau after one hour of incubation, at about the same time as the TCA-soluble radioactivity increased significantly in the medium. The degradation was strongly inhibited by the weak base chloroquine and the proteinase inhibitor leupeptin (FIG. 5C), which both inhibit lysosomal proteolysis. The uptake was mediated by FIBCD1 as no uptake was seen in non-transfected CHO cells (FIG. 5B). Furthermore the uptake and degradation of ¹²⁵I labelled acetylated BSA was inhibited by 10 mM GlcNAc, unlabelled acetylated BSA and by the monoclonal antibody HG-HYB-12-1 that specifically inhibits the binding between NAc-BSA and FIBCD1 (FIG. 5D). Finally the inventors showed, that CHO cells transfected with FIBCD1 cDNA mediate uptake of Alexa488 labelled acetylated BSA while no uptake was seen in non-transfected CHO cells (FIG. 4E).

Immunohistochemical localization of FIBCD1

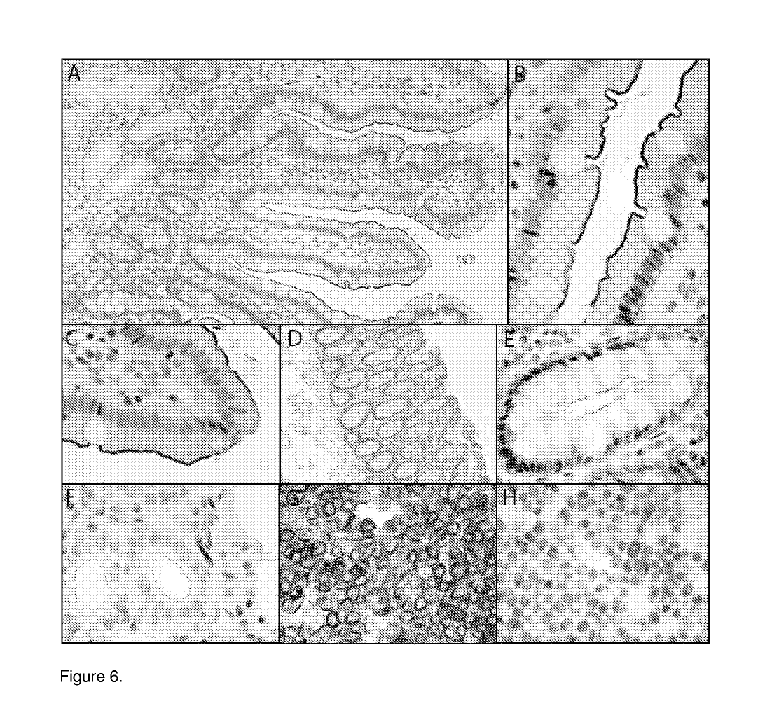

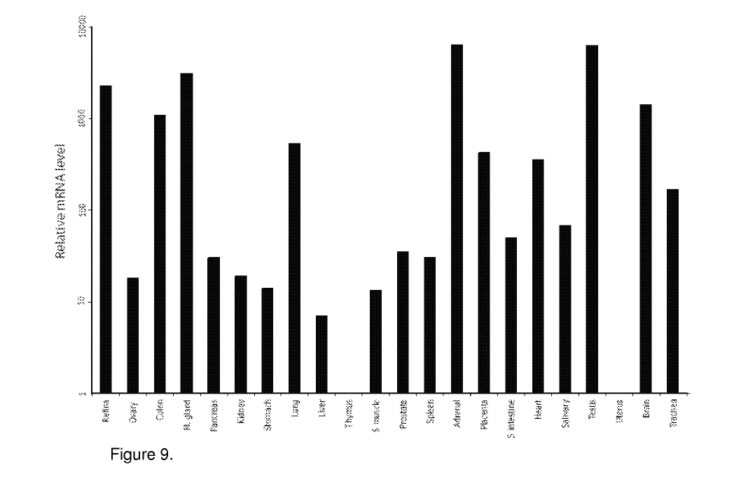

The tissue distribution of FIBCD1 is unknown and the inventors therefore examined the FIBCD1 localization by immunohistochemistry (FIG. 6) and RT-PCR (FIG. 9). Immunoreactivity was found in small and large intestine epithelial cells with a highly polarized localization to the apical surface corresponding to the brush border (FIG. 6 A-E) and in the ducts of the salivary glands (FIG. 6E). Weak or no reactivity was observed in stomach epithelial cells, in respiratory urogenital epithelial cells (not shown). Strong immunoreactivity was found in HEK293 transfected with FIBCD1 cNDA expressing while no reaction was found in non-transfected HEK293 cells (FIG. 6 G, H). The specificity of the monoclonal antibody used for immunohistochemical analysis was further analysed using triton-X100 lysates of HEK293 and HEK293 transfected with FIBCD1 cDNA for Western blotting (FIG. 10).

The inventors then looked for localization of the acid mammalian chitinase (AMAse) in the gut using immunohistochemistry. The inventors found weak immunoreactivity in the epithelial cells of the stomach and strong immunoreactivity in the Paneth cells of the small intestine. No reaction was seen in the epithelial cells of the large intestine or in salivary glands. The Paneth cells are important effecter cells of the small intestine. They are located at the crypt base and they secrete important antimicrobial proteins stored in abundant secretory granules. The antimicrobial proteins include the α-defencins and the C-type lectin HIP/PAP.

The relative levels of FIBCD1 mRNA was determined in 22 different human tissues by real time PCR (FIG. 9). The highest levels of FIBCD1 expression were found in testis, adrenal gland, brain, mammary gland, retina, placenta, colon and lung. No clear correlation was found between the tissue distribution found by RT-PCR and immunohistochemistry. A panel of different cell lines was also analysed by RT-PCR. In general low FIBCD1 mRNA expression was found in all cell lines, but the highest mRNA expression was found in the epithelial cell lines cell lines HUTU and MCF₇ (not shown).

### Diseases Treated

It has been hypothesized that the allergic response is a misdirected immune response against helminth infections. Chitin has recently been implicated as a pathogen-associated molecular pattern (PAMP) that modulates the allergic response (Reese T A, et al. Nature, 2007, 447:92-96).

### Allergic Diseases

It is well known that for example genetically predisposed individuals become sensitised (allergic) to allergens originating from a variety of environmental sources, of which the individuals are exposed. The allergic reaction may occur when a previously sensitised individual is re-exposed to the same or a homologous allergen. Allergic responses range from hay fever, rhinoconductivitis, rhinitis and asthma to systemic anaphylaxis and death in response to e.g. bee or hornet sting or insect bite. The reaction is immediate and can be caused by a variety of atopic allergens such as compounds originating from grasses, trees, weeds, insects, (house dust) mites, food, drugs, chemicals and perfumes.

In one aspect of the present invention, the group of allergic diseases comprises the following, non-limiting, diseases: allergic asthma, asthma, extrinsic bronchial asthma, chronic obstructive pulmonary disease, hay fever (seasonal allergic rhinitis), allergic rhinitis, allergic conjunctivitis, hives, eczema, urticaria, angioedema, onchocercal dermatitis, atopic dermatitis, dermatitis, swelling, hypersensitivity pneumonitis, bronchopulmonary dysplasia, food allergy, perfume allergy, drug allergy, and/or other allergic diseases.

The most common allergens, which cause allergic reactions, include inhalation allergens originating from trees, herbs, weed, plants, grasses, fungi, house dust mites, storage mites, cockroaches and animal hair, feathers, and dandruff. Important pollen allergens from trees, grasses, weeds and herbs are such originating from the taxonomic orders of Fagales, Oleales and Pinales including birch (Betula), alder (Alnus), hazel (Corylus), hornbeam (Carpinus) and olive (Olea), the order of Poales including grasses of the genera Lolium, Phleum, Poa, Cynodon, Dactylis and Secale, the orders of Asterales and Urticales including i. a. herbs of the genera Ambrosia and Artemisia. Important inhalation allergens from fungi are i.a. such originating from the genera Alternaria and Cladosporium. Other important inhalation allergens are those from house dust mites of the genus Dermatophagoides, storage mites from the genus Lepidoglyphys destructor, those from cockroaches and those from vertebrates such as cat, dog, horse, cow, and bird. Also, allergic reactions towards stinging or biting insects such as those from the taxonomic order of Hymenoptera including bees, wasps, and ants are commonly observed. Specific allergen components are known to the person skilled in the art and include e.g. Bet v 1 (B. verrucosa, birch), Aln g 1 (Alnus glutinosa, alder), Cor a 1 (Corylus avelana, hazel) and Car b 1 (Carpinus betulus, hornbeam) of the Fa-gales order. Others are Cry j 1 (Pinales), Amb a 1 and 2, Art v 1 (Asterales), Par j 1 (Urticales), Ole e 1 (Oleales), Ave e 1, Cyn d 1, Dac g 1, Fes p 1, Hol l 1, Lol p 1 and 5, Pas n 1, Phl p 1 and 5, Poa p 1, 2 and 5, Sec c 1 and 5, and Sor h 1 (various grasspollens), Alt a 1 and Cla h 1 (fungi), Der f 1 and 2, Der p 1 and 2 (house dust mites, D. farinae and D. pteronyssinus, respectively), Lep d 1, Bla g 1 and 2, Per a 1 (cockroaches, Blatella germanica and Periplaneta americana, respectively), Fel d 1 (cat), Can f 1 (dog), Equ c 1, 2 and 3 (horse), Apis m 1 and 2 (honeybee), Ves g 1, 2 and 5, Pol a 1, 2 and 5 (all wasps) and Sol i 1, 2, 3 and 4 (fire ant), to mention the most common.

### Inflammatory Bowel Diseases

Inflammatory bowel disease (IBD) is a group of inflammatory conditions of the large and small intestine. The two major types of IBD are Crohn's disease and ulcerative colitis (UC). The main difference between Crohn's disease and UC is the location and nature of the inflammatory changes. Crohn's can affect any part of the gastrointestinal tract, from mouth to anus (skip lesions), although a majority of the cases start in the terminal ileum. Ulcerative colitis, in contrast, is restricted to the colon and the rectum.

Crohn's disease occurs when the lining and wall of the intestines become inflamed and ulcers develop. Although Crohn's disease can occur in any part of the digestive system, it often occurs in the lower part of the small intestine where it joins the colon.

The intestine becomes inflamed, meaning the lining of the intestinal wall reddens and swells. It can become irritated, causing it to bleed and preventing it from properly absorbing the nutrients from digested food. Some people with Crohn's disease have minor symptoms and hardly any discomfort or pain. Their symptoms may only flare a few times. But others may experience frequent diarrhea, intestinal ulcers, and problems in other parts of their bodies, such as inflammation of the joints, skin rashes, and eye problems. Crohn's disease can cause the intestines to become blocked by swelling and scar tissue. People with the condition may also be more susceptible to infections and developing abscesses in and around their intestines.

In ulcerative colitis, the large intestine becomes inflamed and ulcers may develop. Ulcerative colitis affects only the large intestine. The inflammation begins in the rectum (the last few inches of the large intestine where faeces are stored before they leave the body) and can affect only the rectum or the part of the large intestine that joins it. However, most kids and teens, which have ulcerative colitis have the condition throughout their large intestines.

In one aspect of the present invention, the group of inflammatory bowel diseases comprises the following, non-limiting, diseases: Crohn's disease, ulcerative colitis, collagenous colitis, lymphocytic colitis, ischaemic colitis, diversion colitis, Behcet's syndrome, infective colitis, indeterminate colitis, and/or other inflammatory bowel diseases.

Active Substances (“FIBCD1 Binding Molecules” and “Non-FIBCD1 Binding Molecules”)

In one aspect of the invention the pharmaceutical composition of the invention comprises one or more FIBCD1 binding molecules or pharmaceutically acceptable salts, esters, and amides thereof.

In another aspect of the invention the pharmaceutical composition of the invention comprises one or more FIBCD1 binding molecules or pharmaceutically acceptable salts, esters, and amides thereof wherein said one or more FIBCD1 binding molecules is used for the prevention or treatment of diseases.

In yet another aspect of the invention the pharmaceutical composition of the invention comprises one or more FIBCD1 binding molecules or pharmaceutically acceptable salts, esters, and amides thereof wherein said one or more FIBCD1 binding molecules is used for the prevention or treatment of allergic diseases and/or inflammatory bowel diseases.

In another aspect of the invention the pharmaceutical composition of the invention comprises one or more FIBCD1 binding molecules or pharmaceutically acceptable salts, esters, and amides thereof and one or more non-FIBCD1 binding molecules or pharmaceutically acceptable salts, esters, and amides thereof. In yet another aspect of the invention the pharmaceutical composition of the invention comprises one or more FIBCD1 binding molecules or pharmaceutically acceptable salts, esters, and amides thereof and one or more non-FIBCD1 binding molecules or pharmaceutically acceptable salts, esters, and amides thereof wherein said one or more FIBCD1 binding molecules and/or one or more non-FIBCD1 binding molecules is used for the prevention or treatment of diseases.

In yet another aspect of the invention the pharmaceutical composition of the invention comprises one or more FIBCD1 binding molecules or pharmaceutically acceptable salts, esters, and amides thereof and one or more non-FIBCD1 binding molecules or pharmaceutically acceptable salts, esters, and amides thereof wherein said one or more FIBCD1 binding molecules and/or one or more non-FIBCD1 binding molecules is used for the prevention or treatment of an allergic diseases and/or inflammatory bowel diseases.

In an embodiment of the invention the pharmaceutical composition of the invention comprises one or more FIBCD1 binding molecules containing one or more acetyl groups, acetate or is an acetyl radical or pharmaceutically acceptable salts, esters, and amides of said one or more FIBCD1 binding molecules.

In a further embodiment of the invention the pharmaceutical composition of the invention comprises one or more FIBCD1 binding molecules or pharmaceutically acceptable salts, esters, and amides thereof selected from: sialic acid, sialic acid dimer, sialic acid trimer, sialic acid tetramer, sialic acid pentamer, sialic acid hexamer, N-acetylglucosamine, N-acetylglucosamine dimer, N-acetylglucosamine trimer, N-acetylglucosamine tetramer, N-acetylglucosamine pentamer, N-acetylglucosamine hexamer, N-acetylmannosamine, N-acetylmannosamine dimer, N-acetylmannosamine trimer, N-acetylmannosamine tetramer, N-acetylmannosamine pentamer, N-acetylmannosamine hexamer, N-acetylgalactosamine, N-acetylgalactosamine dimer, N-acetylgalactosamine trimer, N-acetylgalactosamine tetramer, N-acetylgalactosamine pentamer, N-acetylgalactosamine hexamer, N-acetylalanine, N-acetylalanine dimer, N-acetylalanine trimer, N-acetylalanine tetramer, N-acetylalanine pentamer, N-acetylalanine hexamer, acetylated carbohydrates, N-acetylated carbohydrates, acetylated D- and L-amino acids, N-acetylated D- and L-amino acids, acetylated nucleic acids, acetylcholine, chitin and, where possible, partially deacetylated analogous thereof or pharmaceutically acceptable salts, esters, and amides thereof.

In a further embodiment of the invention, monomers, dimers, trimers, tetramers, pentamers, hexamers and longer oligomers, polymers and, where possible, partially deacetylated analogous of D- and L-sialic acid, N-acetyl-D- and L-glucosamine, N-acetyl-D- and L-mannosamine, N-acetyl-D- and L-galactosamine, D- and L-N-acetylalanine, acetylated D- and L-carbohydrates, N-acetylated D- and L-carbohydrates, acetylated D- and L-amino acids, N-acetylated D- and L-amino acids, acetylated natural nucleic acids, acetylated non-natural nucleic acids, acetylcholine, chitin, or mixtures thereof are also claimed. Furthermore, the different monomers, dimers, trimers, tetramers, pentamers, hexamers and longer oligomers and polymers of the acetylated compounds mentioned herein may be linked together in any spatial orientation possible. For example, the acetylated carbohydrate based compounds may be linked together by α-1,3, α-1,4, α-1,6, β-1,3, β-1,4, β-1,6 glycosidic linkages or mixtures thereof. The glycosidic linkages may further be selected from S-glycosidic bonds, N-glycosidic bonds, O-glycosidic bonds or mixtures thereof.

In another aspect of the pharmaceutical composition of the invention the one or more non-FIBCD1 binding molecules or pharmaceutically acceptable salts, esters, and amides thereof, which is used together with one or more FIBCD1 binding molecules, is a drug.

In an embodiment of the pharmaceutical composition of the invention the one or more non-FIBCD1 binding molecules or pharmaceutically acceptable salts, esters, and amides thereof, which is used together with one or more FIBCD1 binding molecules, is a drug selected from the group comprising anti-asthma drugs, anti-allergy drugs, anti-histamine drugs, smooth muscle cell relaxing drugs, mast-cell stabilizers, anti-IgE drugs, selective or non-selective potassium channel activators (bronchodilators), immunomodulating agents, mucus secretion inhibitors, mucus liquefying agents, leukotriens modifier drugs, leukotriens receptor antagonists, mesalamine, steroids, prednisone, Asacol, and Immunosuppressants such as prednisone, infliximab (Remicade), azathioprine (Imuran), methotrexate, or 6-mercaptopurine.

In another aspect of the pharmaceutical composition of the invention the one or more non-FIBCD1 binding molecules or pharmaceutically acceptable salts, esters, and amides thereof, which is used together with one or more FIBCD1 binding molecules, are effective to treat a subject to prevent the development of allergic diseases, which subject is diagnosed in having an increased risk for the development of allergic diseases.

In yet another aspect of the pharmaceutical composition of the invention the one or more non-FIBCD1 binding molecules or pharmaceutically acceptable salts, esters, and amides thereof, which is used together with one or more FIBCD1 binding molecules, are effective to treat a subject to prevent the development of inflammatory bowel diseases, which subject is diagnosed in having an increased risk for the development of inflammatory bowel diseases.

In an embodiment of the pharmaceutical composition of the invention the one or more FIBCD1 binding molecules or pharmaceutically acceptable salts, esters, and amides thereof comprises one or more antibodies that inhibits or stimulates the function of the naturally occurring FIBCD1.

In another embodiment of the pharmaceutical composition of the invention the one or more FIBCD1 binding molecules or pharmaceutically acceptable salts, esters, and amides thereof comprises one or more antisense oligonucleotides that binds to the mRNA and/or DNA of the naturally occurring FIBCD1 or a part thereof and inhibits the transcription and/or the translation of FIBCD1.

An antisense oligonucleotide according to the invention can be, for example, about 5 to 10, 11 to 20, 21 to 30, 31 to 40, or 41 to 50 nucleotides in length. The length of the antisense oligonucleotide used according to the present invention depends on the monomers which are present in the oligonucleotide. The length of the oligonucleotide should be long enough to ensure specific binding to the target sequence and the sequence should be long enough to ensure that the antisense oligonucleotide forms: 1) a stable double helix if the target is mRNA, or 2) that it forms a stable triple helix if the target is DNA.

In another embodiment of the pharmaceutical composition of the invention the one or more antisense oligonucleotides comprises nine or more monomers, such as about 9-20, about 20-30, about 30-40, or about 40-50 monomers individually selected from the group of nucleic acid monomers comprising DNA, RNA, PNA, LNA and/or phosphorothioate.

In another aspect of the pharmaceutical composition of the invention the one or more FIBCD1 binding molecules or pharmaceutically acceptable salts, esters, and amides thereof are covalently or non-covalently attached to said non-FIBCD1 binding molecule or pharmaceutically acceptable salts, esters, and amides thereof.

In another embodiment of the invention the pharmaceutical composition, which comprises one or more FIBCD1 binding molecules and optionally one or more non-FIBCD1 binding molecules, is suitable for the treatment of subjects having allergic asthma, asthma, extrinsic bronchial asthma, chronic obstructive pulmonary disease, hay fever (seasonal allergic rhinitis), allergic rhinitis, allergic conjunctivitis, hives, eczema, urticaria, angioedema, onchocercal dermatitis, atopic dermatitis, dermatitis, swelling, hypersensitivity pneumonitis, bronchopulmonary dysplasia, food allergy, and/or other allergic diseases.

In yet another embodiment of the invention the pharmaceutical composition, which comprises one or more FIBCD1 binding molecules and optionally one or more non-FIBCD1 binding molecules, is suitable for the treatment of subjects having inflammatory bowel disease such as Crohn's disease, ulcerative colitis, collagenous colitis, lymphocytic colitis, ischaemic colitis, diversion colitis, Behcet's syndrome, infective colitis, indeterminate colitis, and/or other inflammatory bowel diseases.

In another embodiment of the invention the pharmaceutical composition, which comprises one or more FIBCD1 binding molecules and optionally one or more non-FIBCD1 binding molecules, is formulated for topical, oral, aerosol, intranasal, intraarterial, intradermal, intravesical, intraocular, parenteral, subcutaneous, intrathecal, intravenous, intramuscular, interperitoneal, rectal, vaginal or transdermal administration.

In further embodiments, the use of the above mentioned pharmaceutical compositions, which comprises one or more FIBCD1 binding molecules and optionally one or more non-FIBCD1 binding molecules, for the manufacture of a medicament for treating or preventing allergic diseases and/or inflammatory bowel diseases are claimed.

In further embodiments, methods for treating or preventing allergic diseases and/or inflammatory bowel diseases comprising administering to a subject an effective dose of the above mentioned pharmaceutical compositions are also claimed.

FIBCD1 binding molecules encompass, but are not limited to, antibodies and fragments thereof, antisense nucleic acid molecules, peptides, and low molecular weight non-peptide molecules, such as acetylated carbohydrates, binding to the FIBCD1 antigen.

In a particular preferred embodiment of the invention the FIBCD1 binding molecules comprise acetylated compounds.

### Acetylated Compounds

Other FIBCD1 binding molecules or pharmaceutically acceptable salts, esters, and amides thereof according to the present invention, i.e. compounds which binds to—or otherwise modulates the effect of the naturally occurring FIBCD1 comprise: monomers, dimers, trimers, tetramers, pentamers, hexamers and longer oligomers, polymers and, where possible, partially deacetylated analogous of sialic acid, N-acetylglucosamine, N-acetylmannosamine, N-acetylgalactosamine, N-acetylalanine, acetylated carbohydrates, acetylated D- and L-amino acids, acetylated natural nucleic acids, acetylated non-natural nucleic acids, acetylcholine or mixtures thereof are also claimed. Furthermore, the different monomers, dimers, trimers, tetramers, pentamers, hexamers and longer oligomers and polymers of the acetylated compounds mentioned herein may be linked together in any spatial orientation possible. For example, each acetylated carbohydrate and/or N-acetylated carbohydrate based monomer may be linked together by α-1,3, α-1,4, α-1,6, β-1,3, β-1,4, β-1,6 glycosidic linkages or mixtures thereof. The glycosidic linkages may further be selected from S-glycosidic bonds, N-glycosidic bonds, O-glycosidic bonds or mixtures thereof. Longer oligomers and polymers may comprise at least 7 monomer units, such as 7-20, 21 to 30, 31-40, 41-50, 51-60, 61-70, 71-80, 81-90, 91-100, 100-200, 200-300, 300-400, 400-500, 500-1000, 1000-2000 or more monomer units.

In another aspect of the invention the FIBCD1 binding molecules or pharmaceutically acceptable salts, esters, and amides thereof comprise: sialic acid, sialic acid dimer, sialic acid trimer, sialic acid tetramer, sialic acid pentamer, sialic acid hexamer, N-acetylglucosamine, N-acetylglucosamine dimer, N-acetylglucosamine trimer, N-acetylglucosamine tetramer, N-acetylglucosamine pentamer, N-acetylglucosamine hexamer, N-acetylmannosamine, N-acetylmannosamine dimer, N-acetylmannosamine trimer, N-acetylmannosamine tetramer, N-acetylmannosamine pentamer, N-acetylmannosamine hexamer, N-acetylgalactosamine, N-acetylgalactosamine dimer, N-acetylgalactosamine trimer, N-acetylgalactosamine tetramer, N-acetylgalactosamine pentamer, N-acetylgalactosamine hexamer, N-acetylalanine, N-acetylalanine dimer, N-acetylalanine trimer, N-acetylalanine tetramer, N-acetylalanine pentamer, N-acetylalanine hexamer, acetylated carbohydrates, acetylated amino acids, acetylated nucleic acids, acetylcholine, chitin, acetate, an acetyl radical and, where possible, partially deacetylated analogous or mixtures thereof. Additional non-limiting examples of a “FIBCD1 binding molecule” comprise, where possible, also longer oligomers and polymers of the herein mentioned acetylated compounds comprising at least 7 monomer units, such as 7-20, 21 to 30, 31-40, 41-50, 51-60, 61-70, 71-80, 81-90, 91-100, 100-200, 200-300, 300-400, 400-500, 500-1000, 1000-2000 or more monomer units. Thus oligomers and polymers according to the present invention comprise compounds consisting of about 7-20, 21 to 30, 31-40, 41-50, 51-60, 61-70, 71-80, 81-90, 91-100, 100-200, 200-300, 300-400, 400-500, 500-1000, 1000-2000 or more monomer units.

In a further aspect of the invention the acetylated FIBCD1 binding molecules or pharmaceutically acceptable salts, esters, and amides thereof may comprise: sialic acid, sialic acid dimer, sialic acid trimer, sialic acid tetramer, sialic acid pentamer, sialic acid hexamer, N-acetylglucosamine, N-acetylglucosamine dimer, N-acetylglucosamine trimer, N-acetylglucosamine tetramer, N-acetylglucosamine pentamer, N-acetylglucosamine hexamer, N-acetylmannosamine, N-acetylmannosamine dimer, N-acetylmannosamine trimer, N-acetylmannosamine tetramer, N-acetylmannosamine pentamer, N-acetylmannosamine hexamer, N-acetylgalactosamine, N-acetylgalactosamine dimer, N-acetylgalactosamine trimer, N-acetylgalactosamine tetramer, N-acetylgalactosamine pentamer, N-acetylgalactosamine hexamer, N-acetylalanine, N-acetylalanine dimer, N-acetylalanine trimer, N-acetylalanine tetramer, N-acetylalanine pentamer, N-acetylalanine hexamer, acetylated carbohydrates, acetylated amino acids, acetylated nucleic acids, acetylcholine, chitin, acetate, an acetyl radical or mixtures thereof as well as, where possible, partially deacetylated analogous. Additional non-limiting examples of a “FIBCD1 binding molecule” comprise, where possible, also longer oligomers and polymers of the herein mentioned acetylated compounds comprising at least 7 monomer units, such as 7-20, 21 to 30, 31-40, 41-50, 51-60, 61-70, 71-80, 81-90, 91-100, 100-200, 200-300, 300-400, 400-500, 500-1000, 1000-2000 or more monomer units. Thus oligomers and polymers according to the present invention comprise compounds consisting of about 7-20, 21 to 30, 31-40, 41-50, 51-60, 61-70, 71-80, 81-90, 91-100, 100-200, 200-300, 300-400, 400-500, 500-1000, 1000-2000 or more monomer units.

### Antibodies

The anti-FIBCD1 antibody, according to the present invention, may be mono-, bi- or multispecific. Indeed, bispecific antibodies, diabodies, and the like, provided by the present invention may bind any suitable target in addition to a portion of FIBCD1. As indicated above, the term “antibody” as used herein, unless otherwise stated or clearly contradicted by the context, includes fragments of an antibody provided by any known technique, such as enzymatic cleavage, peptide synthesis and recombinant techniques that retain the ability to specifically bind to an antigen. It has been shown that the antigen-binding function of an antibody may be performed by fragments of a full-length (intact) antibody. Examples of antigen-binding fragments encompassed within the term “antibody” include, but are not limited to (i) a Fab fragment, a monovalent fragment consisting of the V_{L}, V_{H}, C_{L} and C_{H}1 domains; (ii) F(ab)₂ and F(ab′)₂ fragments, bivalent fragments comprising two Fab fragments linked by a disulfide bridge at the hinge region; (iii) a Fd fragment consisting essentially of the V_{H} and C_{H}1 domains; (iv) a Fv fragment consisting essentially of the V_{L} and V_{H} domains of a single arm of an antibody, (v) a dAb fragment (Ward et al., Nature 341, 544-546 (1989)), which consists essentially of a V_{H} domain and also called domain antibodies (Holt et al. (November 2003) Trends Biotechnol. 21(11):484-90); (vi) a camelid antibody or nanobody (Revets et al. (January 2005) Expert Opin Biol Ther. 5(1):111-24), (vii) an isolated complementarity determining region (CDR), such as a V_{H} CDR3, (viii) a UniBody™, a monovalent antibody as disclosed in WO 2007/059782, (ix) a single chain antibody or single chain Fv (scFv), see for instance Bird et al., Science 242, 423-426 (1988) and Huston et al., PNAS USA 85, 5879-5883 (1988)), (x) a diabody (a scFv dimer), which can be monospecific or bispecific (see for instance PNAS USA 90(14), 6444-6448 (1993), EP 404097 or WO 93/11161 for a description of diabodies), a triabody or a tetrabody.

It should be understood that the term antibody generally includes monoclonal antibodies, polyclonal antibodies as well as parts thereof. The antibodies can be human, humanized, chimeric, murine, etc.

In one embodiment of the present invention, the anti-FIBCD1 antibody is a monoclonal antibody or a fragment thereof.

In a further embodiment of the present invention, the anti-FIBCD1 antibody is a human monoclonal antibody or a fragment thereof.

In a further embodiment of the present invention, the anti-FIBCD1 antibody is a humanized monoclonal antibody or a fragment thereof.

In a further embodiment of the present invention, the anti-FIBCD1 antibody is a chimeric monoclonal antibody or a fragment thereof.

In a further embodiment of the present invention, the anti-FIBCD1 antibody is a murine monoclonal antibody or a fragment thereof.

In another embodiment of the present invention, the anti-FIBCD1 antibody is a polyclonal antibody or a fragment thereof.

In a further embodiment of the present invention, the anti-FIBCD1 antibody is a human polyclonal antibody or a fragment thereof.

In a further embodiment of the present invention, the anti-FIBCD1 antibody is a humanized polyclonal antibody or a fragment thereof.

In a further embodiment of the present invention, the anti-FIBCD1 antibody is a chimeric polyclonal antibody or a fragment thereof.

In a further embodiment of the present invention, the anti-FIBCD1 antibody is a murine polyclonal antibody or a fragment thereof.

In further embodiments, the use of the above mentioned anti-FIBCD1 antibodies for the manufacture of a medicament for treating or preventing diseases are claimed.

In further embodiments, the use of the above mentioned anti-FIBCD1 antibodies for the manufacture of a medicament for treating or preventing allergic diseases and/or inflammatory bowel diseases are claimed.

In further embodiments, the use of the above mentioned anti-FIBCD1 antibodies for the manufacture of a medicament for treating or preventing allergic wherein the allergic disease is selected from the group comprising: allergic asthma, asthma, extrinsic bronchial asthma, chronic obstructive pulmonary disease, hay fever (seasonal allergic rhinitis), allergic rhinitis, allergic conjunctivitis, hives, eczema, urticaria, angioedema, onchocercal dermatitis, atopic dermatitis, dermatitis, swelling, hypersensitivity pneumonitis, bronchopulmonary dysplasia, food allergy, perfume allergy, drug allergy, and/or other allergic diseases.

In further embodiments, the use of the above mentioned anti-FIBCD1 antibodies for the manufacture of a medicament for treating or preventing inflammatory bowel diseases, wherein the inflammatory bowel disease is selected from the group comprising: Crohn's disease, ulcerative colitis, collagenous colitis, lymphocytic colitis, ischaemic colitis, diversion colitis, Behcet's syndrome, infective colitis, indeterminate colitis, and/or other inflammatory bowel diseases

In further embodiments, methods for treating or preventing allergic diseases and/or inflammatory bowel diseases comprising administering to a subject an effective dose of the above mentioned anti-FIBCD1 antibodies are also claimed.

In further embodiments, methods for treating or preventing allergic asthma, asthma, extrinsic bronchial asthma, chronic obstructive pulmonary disease, hay fever (seasonal allergic rhinitis), allergic rhinitis, allergic conjunctivitis, hives, eczema, urticaria, angioedema, onchocercal dermatitis, atopic dermatitis, dermatitis, swelling, hypersensitivity pneumonitis, bronchopulmonary dysplasia, food allergy, perfume allergy, drug allergy, and/or other allergic diseases comprising administering to a subject an effective dose of the above mentioned anti-FIBCD1 antibodies are also claimed.

In further embodiments, methods for treating or preventing allergic Crohn's disease, ulcerative colitis, collagenous colitis, lymphocytic colitis, ischaemic colitis, diversion colitis, Behcet's syndrome, infective colitis, indeterminate colitis, and/or other inflammatory bowel diseases comprising administering to a subject an effective dose of the above mentioned anti-FIBCD1 antibodies are also claimed.

In all the above embodiments, the anti-FIBCD1 antibody specifically binds to the naturally occurring FIBCD1 receptor or a part thereof.

### Antisense Nucleic Acid Molecules

In one embodiment, an inhibitory compound of the invention is an antisense nucleic acid molecule that is complementary to a gene encoding FIBCD1, or to a portion of said gene, or a recombinant expression vector encoding said antisense nucleic acid molecule. The antisense nucleic acid molecule may comprise a DNA, a RNA, a PNA, a LNA, a phosphorothioate or mixtures thereof. The use of antisense nucleic acid molecules to downregulate the expression of a particular protein in a cell is well known in the art. An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid molecule may be complementary in sequence to a regulatory region of the gene encoding the mRNA, for instance a transcription initiation sequence or regulatory element.

Preferably, an antisense oligonucleotide, according to the invention, is designed so as to be complementary to a region preceding or spanning the initiation codon on the coding strand or in the 3′ untranslated region of an mRNA- or DNA strand.

Given the known nucleotide sequences for the coding strands of the FIBCD1 gene (and thus the known sequences of the FIBCD1 mRNA), antisense nucleic acid molecules of the invention can be designed according to the rules of Watson and Crick base pairing. The antisense nucleic acid molecule can be complementary to the entire coding region of the FIBCD1 mRNA, but more preferably is an oligonucleotide which is antisense to only a portion of the coding or noncoding region of the FIBCD1 mRNA. For example, the antisense oligonucleotide can be complementary to the region surrounding the translation start site of the FIBCD1 mRNA. An antisense oligonucleotide can be, for example, about 5, 10, 15, 20, 25, 30, 35, 40, 45 or 50 nucleotides in length, such as 5-10, 10-20, 20-30, 30-40, or 40-50 nucleotides in length. An antisense nucleic acid molecule of the invention can be constructed using chemical synthesis and enzymatic ligation reactions using procedures known in the art. For example, an antisense nucleic acid molecule (e.g. an antisense oligonucleotide) can be chemically synthesized using naturally occurring nucleotide monomers or variously modified nucleotide monomers designed to increase the biological stability of the molecules or to increase the physical stability of the duplex formed between the antisense and sense nucleic acid molecules, e.g., phosphorothioate derivatives and acridine substituted nucleotides can be used.

Examples of modified nucleotides which can be used to generate the antisense nucleic acid monomers include LNA nucleotides such as α- or β-D-ribo-LNAs, α- or β-D-xylo-LNAs, α- or β-L-LNAs, α- or β-2′-Thio-LNAs, α- or β-2′-Amino-LNAs, α- or β-phosphorthioate LNAs, α- or β-methylphosphonate LNAs, α- or β-amidate LNAs and PNA nucleotides as well as 5-fluorouracil, 5-bromouracil, 5-chlorouracil, 5-iodouracil, hypoxanthine, xanthine, 4-acetylcytosine, 5-(carboxyhydroxylmethyl)-uracil, 5-carboxymethylaminmethyl-2-thiouridine, 5-carboxymethylaminomethyl-uracil, dihydrouracil, β-D-galactosylqueosine, inosine, N-6-isopentenyladenine, 1-methylguanine, I-methylinosine, 2,2-dimethylguanine, 2-methyladenine, methylguanine, 3-methylcytosine, 5-methylcytosine, N-6-adenine, 7-methylguanine, 5-methylaminomethyluracil, 5-methoxyaminomethyl-2-thiouracil, β-D-mannosylqueosine, 5′-methoxycarboxymethyl-uracil, 5-methoxyu racil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)-uracil, (acp3)w, and 2,6-diaminopurine. To inhibit FIBCD1 expression in cells in culture, one or more antisense oligonucleotides can be added to the culture media.

Alternatively, an antisense oligonucleotide can be produced biologically using an expression vector into which all or a portion of FIBCD1 cDNA has been subcloned in an antisense orientation (i.e., nucleic acid transcribed from the inserted nucleic acid will be of an antisense orientation to a target nucleic acid of interest). Regulatory sequences operatively linked to a nucleic acid cloned in the antisense orientation can be chosen which direct the expression of the antisense RNA molecule in a cell of interest, for instance promoters and/or enhancers or other regulatory sequences can be chosen which direct constitutive, tissue specific or inducible expression of antisense RNA. The antisense expression vector is prepared according to standard recombinant DNA methods for constructing recombinant expression vectors, except that the FIBCD1 cDNA (or portion thereof) is cloned into the vector in the antisense orientation. The antisense expression vector can be in the form of, for example, a recombinant plasmid, phagemid or attenuated virus. The antisense expression vector is introduced into cells using a standard transfection technique.

The antisense nucleic acid molecules of the invention are typically administered to a subject or generated in situ such that they hybridize with or bind to cellular mRNA and/or genomic DNA encoding the FIBCD1 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of an antisense nucleic acid molecule of the invention includes direct injection at a tissue site.

Alternatively, an antisense nucleic acid molecule can be modified to target selected cells and then administered systemically. For example, for systemic administration, an antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense nucleic acid molecules, vector constructs in which the antisense nucleic acid molecule is placed under the control of a strong pol 11 or pol III promoter are preferred.

In yet another embodiment, the antisense nucleic acid molecule of the invention is ana-anomeric nucleic acid molecule. Ana-anomeric nucleic acid molecule forms specific double-stranded hybrids with complementary RNA in which, contrary to the usual P-units, the strands run parallel to each other.

The antisense nucleic acid molecule can also comprise a2′-O-methylribonucleotide or achimeric RNA-DNA analogue.

In still another aspect, an antisense nucleic acid molecule of the invention is a riboyzyme. Ribozymes are catalytic RNA molecules withribonuclease activity which are capable of cleaving a single-stranded nucleic acid, such as an mRNA, to which they have a complementary region. Thus, ribozymes (e.g., hammerhead ribozymes) can be used to catalytically cleave FIBCD1 mRNA transcripts to thereby inhibit translation of FIBCD1 mRNA. A ribozyme having specificity for the FIBCD1-encoding nucleic acid can be designed based upon the nucleotide sequence of the FIBCD1 cDNA.

For example, a derivative of a Tetrahymena L-19 IVS RNA can be constructed in which the nucleotide sequence of the active site is complementary to the nucleotide sequence to be cleaved in the FIBCD1-encoding mRNA.

Alternatively, FIBCD1 gene expression can be inhibited by targeting nucleotide sequences complementary to a regulatory region of the FIBCD1 gene (e.g., a FIBCD1 promoter and/or enhancer) to form triple helical structures that prevent transcription of an FIBCD1 gene in target cells.

In one embodiment of the invention the pharmaceutical composition of the invention comprises one or more antisense nucleic acid molecules (antisense oligonucleotides) that specifically binds to FIBCD1 or a part thereof.

An antisense oligonucleotide according to the invention can be, for example, about 5 to 10, 11 to 20, 21 to 30, 31 to 40, or 41 to 50 nucleotides in length. The length of the antisense oligonucleotide used according to the present invention depends on the monomers which are present in the oligonucleotide. The length of the oligonucleotide should be long enough to ensure specific binding to the target sequence and long enough to ensure that the antisense oligonucleotide forms, if the target is mRNA, a stable duplex or, if the target is DNA, that it forms a stable triplex.

An embodiment of the invention comprises a single stranded antisense oligonucleotide, or a conjugate thereof, having a length of 9 to 50 nucleobase units, wherein said antisense oligonucleotide is complementary to at least a part of the mRNA-nucleotide sequence found in SEQ ID No 3.

In another embodiment of the invention the pharmaceutical composition of the invention comprises one or more antisense oligonucleotides, said antisense oligonucleotide comprising nine or more monomers individually selected from the group of nucleic acid monomers comprising DNA, RNA, PNA, LNA and/or phosphorothioate.

Another embodiment of the present invention comprises an antisense oligonucleotide that is complementary to a mRNA and/or a DNA molecule encoding the naturally occurring FIBCD1 receptor or a part thereof.

Yet another embodiment of the present invention comprises a single stranded antisense oligonucleotide, or a conjugate thereof, according to claim 32, wherein said oligonucleotide comprise the sequence:

| | |
|---|---|
| 5′-catcttccg-3′ | (SEQ ID No 4) |
| or | |
| 5′-ccatcttccggcaggac-3′ | (SEQ ID No. 5) |

In yet another embodiment of the present invention the antisense oligonucleotide, mentioned above, comprises one or more DNA nucleotides, one or more RNA nucleotides, one or more LNA nucleotides, one or more PNA nucleotides, and/or one or more phosphorothioates.

In further embodiments, the use of the above mentioned antisense oligonucleotides for the manufacture of a medicament for treating or preventing allergic diseases and/or inflammatory bowel diseases are claimed.

In further embodiments, the use of the above mentioned antisense oligonucleotide for the manufacture of a medicament for treating or preventing allergic diseases caused by an allergen selected from the group comprising weed/plant/tree pollens or spores, animal dander, house dust mite, dust, lint, mite feces, fungal spores, wasp-venom, bee-venom, fire ant-venom, penicillin, sulfonamides, food ingredients, perfume, latex and cockroaches are claimed.

In further embodiments, the use of the above mentioned antisense oligonucleotides for the manufacture of a medicament suitable for topical, oral, aerosol, intranasal, intraar-terial, intradermal, intravesical, intraocular, parenteral, subcutaneous, intrathecal, intravenous, intramuscular, interperitoneal, rectal, vaginal or transdermal administration for treating or preventing allergic diseases and/or inflammatory bowel diseases are claimed.

In further embodiments, methods for treating or preventing allergic diseases and/or inflammatory bowel diseases comprising administering to a subject an effective dose of the above mentioned antisense oligonucleotides are also claimed.

### Therapy, Pharmaceutical Compositions and Dosages

The pharmaceutical composition may also comprise a pharmaceutically acceptable carrier, such as a vehicle, an adjuvant, an excipient, or a diluent. Such carriers are well known to the person skilled in the art and are readily available commercially.

The choice of carrier will be determined in part by the particular composition, as well as the particular method used to administer the composition. Accordingly, there is a wide variety of suitable formulations of the pharmaceutical composition of the present invention.

The following formulations for topical, oral, aerosol, intranasal, intraarterial, intradermal, intravesical, intraocular, parenteral, subcutaneous, intrathecal, intravenous, intramuscular, interperitoneal, rectal, vaginal or transdermal administrations are merely exemplary and are in no way limiting.

Formulations for topical application on the skin of the subject or on the subject hair may be in the form of a gel, ointment, emulsion, thick cream, liniment, liquid, balsam, lotion, foam, mask, shampoo, tonic means, cleaner, spray, hair spray, powder including liquid powder, compact powder, cosmetic pencil, or in any other traditional form used in the field of cosmetology or dermatology.

The pharmaceutical composition of the present invention may further comprise one or more drugs or therapeutics as detailed herein and any other additive as disclosed herein.

The pharmaceutical compositions of the present invention may be adapted and/or packaged as a kit for personal use or for use by a medical practitioner, for periodic administration to a subject in doses over any period of time. Typically, for the prevention or treatment of allergies and related diseases or disorders, the period is of 3-30 days, in doses at least once daily up to ten times/day.

The pharmaceutical compositions may be formulated according to conventional pharmaceutical practice, see, e.g., “Remington's Pharmaceutical Sciences” and “Encyclopedia of Pharmaceutical Technology”, edited by Swarbrick, J. & J. C. Boylan, Marcel Dekker, Inc., New York, 1988. Typically the FIBCD1 binding molecules and/or the non-FIBCD1 binding molecules described herein are formulated with at least a pharmaceutically acceptable carrier or excipient. Pharmaceutically acceptable carriers or excipients are those known by the person skilled in the art.

### Oral Formulations

Pharmaceutical compositions for oral use include tablets which contain FIBCD1 binding molecules and/or the non-FIBCD1 binding molecules as described herein, optionally in combination with one or more anti-allergy agents, in admixture with non-toxic pharmaceutically acceptable excipients. These excipients may be, for example, inert diluents or fillers, such as sucrose, sorbitol, sugar, mannitol, microcrystalline cellulose, starches including potato starch, calcium carbonate, sodium chloride, lactose, calcium phosphate, calcium sulfate or sodium phosphate; granulating and disintegrating agents, for example, cellulose derivatives including microcrystalline cellulose, starches including potato starch, croscarmellose sodium, alginates or alginic acid; binding agents, for example, sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone or polyethylene glycol; and lubricating agents, including glidants and antiadhesives, for example, magnesium stearate, zinc stearate, stearic acid, silicas, hydrogenated vegetable oils or talc.

Other pharmaceutically acceptable excipients can be colorants, flavouring agents, plasticisers, humectants, buffering agents, etc.

The tablets may be uncoated or they may be coated by known techniques, optionally to delay disintegration and absorption in the gastrointestinal tract and thereby providing a sustained action over a longer period. The coating may be adapted to release the chemo-sensitising compound in a predetermined pattern, e.g., in order to achieve a controlled release formulation (see below) or it may be adapted not to release the active drug substance until after passage of the stomach (enteric coating). The coating may be a sugar coating, a film coating (e.g. based on hydroxypropyl methylcellulose, methylcellulose, methyl hydroxyethylcellulose, hydroxypropylcellulose, carboxymethylcellulose, acrylate copolymers (EudragitE®), polyethylene glycols and/or polyvinylpyrrolidone) or an enteric coating (e.g. based on methacrylic acid copolymer (Eudragit® L and S), cellulose acetate phthalate, hydroxypropyl methylcellulose phthalate, hydroxypropyl methylcellulose acetate succinate, polyvinyl acetate phthalate, shellac and/or ethylcellulose).

Furthermore, a time delay material such as, e.g., glyceryl monostearate or glyceryl distearate may be employed.

In addition, the solid tablet compositions as mentioned above may be provided with a coating adapted to protect the composition from unwanted chemical changes, e.g. chemical degradation, prior to the release of the FIBCD1 binding molecules and/or the non-FIBCD1 binding molecules.

The coating may be applied on the solid dosage form in a similar manner as that described in “Aqueous film coating” by James A. Seitz in “Encyclopedia of Pharmaceutical Technology”, Vol 1, pp. 337-349 edited by Swarbrick, J. & J. C. Boylan, Marcel Dekker, Inc., New York, 1988.

Formulations for oral use may also be presented as chewing tablets, or as hard gelatin capsules wherein the active ingredient is mixed with an inert solid diluent, for example, potato starch, lactose, microcrystalline cellulose, calcium carbonate, calcium phosphate or kaolin, or as soft gelatin capsules wherein the active ingredient is mixed with water or an oil medium, for example, peanut oil, liquid paraffin, or olive oil.

Powders and granulates may be prepared using the ingredients mentioned above under tablets and capsules in a conventional manner using, e.g., a mixer, a fluid bed apparatus or a spray drying equipment.

Controlled release compositions for oral use may, e.g., be constructed to release the active drug substance by controlling the dissolution and/or the diffusion of the active drug substance.

Dissolution or diffusion controlled release can be achieved by appropriate coating of a tablet, capsule, pellet or granulate formulation of the FIBCD1 binding molecules and/or the non-FIBCD1 binding molecules, or by incorporating the FIBCD1 binding molecules and/or the non-FIBCD1 binding molecules in question in, e.g., an appropriate matrix.

A controlled release coating may comprise one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methyl-methacrylate, 2-hydroxymethacrylate, methacrylate hydrogels, 1,3-butylene glycol, ethylene glycol methacrylate and/or polyethylene glycols.

In a controlled release matrix formulation of the FIBCD1 binding molecules and/or the non-FIBCD1 binding molecules, the matrix material may comprise, e.g., hydrated methylcellulose, carnauba wax and stearyl alcohol, carbopol 934, silicone, glyceryl tristearate, methyl acrylate-methyl methacrylate, polyvinyl chloride, polyethylene and/or halogenated fluorocarbon.

A controlled release composition of the FIBCD1 binding molecules and/or the non-FIBCD1 binding molecules described herein, may also be in the form of a buoyant tablet or capsule, i.e. a tablet or capsule which upon oral administration floats on top of the gastric content for a certain period of time. A buoyant tablet formulation of the FIBCD1 binding molecules and/or the non-FIBCD1 binding molecules in question can be prepared by granulating a mixture of the FIBCD1 binding molecules and/or the non-FIBCD1 binding molecules, excipients and 20-75% w/w of hydrocolloids, such as hydroxyethylcellulose, hydroxypropylcellulose and hydroxypropylmethylcellulose. The obtained granules can then be compressed into tablets.

On contact with the gastric juice, the tablet can form a substantially water-impermeable gel barrier around its surface. This gel barrier takes part in maintaining a density of less than one, thereby allowing the tablet to remain buoyant in the gastric juice.

### Fluid/Liquid Compositions for Oral Use

Powders, dispersible powders or granules suitable for preparation of an aqueous suspension by addition of water are also convenient dosage forms. Formulation as a suspension, an emulsion or a dispersion provides the active substance in admixture with a dispersing or wetting agent, suspending agent, and/or one or more preservatives. Such formulations may also be suitable for use in of an active substance to e.g. a mucosa such as the gastrointestinal, buccal, nasal, rectal, or vaginal mucosa, or for administration to intact or damaged skin, or wounds.

Suitable dispersing or wetting agents are, for example, naturally occurring phosphatides, e.g., lecithin, or soybean lecithin; condensation products of ethylene oxide with e. g. a fatty acid, a long chain aliphatic alcohol, or a partial ester derived from fatty acids and a hexitol or a hexitol anhydride, for example polyoxyethylene stearate, polyoxyethylene sorbitol monooleate, polyoxyethylene sorbitan monooleate etc.

Suitable suspending agents are, e.g., naturally occurring gums such as, e.g., gum acacia, xanthan gum, or gum tragacanth; celluloses such as, e.g., sodium carboxymethylcellulose, microcrystalline cellulose (e.g, Avicel® RC 591, methylcellulose; alginates such as, e,g., sodium alginate, etc.

Suitable examples of preservatives for use in formulations according to the invention are parabens, such as methyl or propyl p-hydroxybenzoate, and benzalkonium chloride.

Rectal and/or Vaginal Formulations

For application to the rectal or vaginal mucosa suitable formulations for use according to the invention include suppositories (emulsion or suspension type), enemas, and rectal gelatin capsules (solutions or suspensions). Appropriate pharmaceutically acceptable suppository bases include cocoa butter, esterified fatty acids, glycerinated gelatin, and various water-soluble or dispersible bases like polyethylene glycols and polyoxyethylene sorbitan fatty acid esters.

Various additives like, e.g., enhancers or surfactants may be incorporated.

### Nasal Formulations

For application to the nasal mucosa, nasal sprays and aerosols for inhalation are suitable compositions for use according to the invention. In a typically nasal formulation, the active substance is present in the form of a particulate formulation optionally dispersed in a suitable vehicle. The pharmaceutically acceptable vehicles and excipients and optionally other pharmaceutically acceptable materials present in the composition such as diluents, enhancers, flavouring agents, preservatives etc. are all selected in accordance with conventional pharmaceutical practice in a manner understood by the persons skilled in the art of formulating pharmaceuticals.

Nasal administration may be employed in those cases where an immediate effect is desired, Furthermore, after administration of a nasal formulation according to the invention, the active substance may be adsorped on the nasal mucosa. The adsorption to the mucosa is believed to lead to a less irritative effect than when e.g. a liquid vehicle e.g. containing a penetration enhancer or promoter is employed.

### Topical Formulations

For application to the skin, the formulations according to the invention may contain conventionally non-toxic pharmaceutically acceptable carriers and excipients including microspheres and liposomes. The formulations include creams, ointments, lotions, liniments, gels, hydrogels, solutions, suspensions, sticks, sprays, pastes, plasters, and other kind of transdermal drug delivery systems. The pharmaceutically acceptable excipients may include emulsifying agents, antioxidants, buffering agents, preservatives, humectants, penetration enhancers, chelating agents, gelforming agents, ointment bases, perfumes, and skin protective agents.

Examples of emulsifying agents are naturally occurring gums, e.g. gum acacia or gum tragacanth, naturally occurring phosphatides, e.g. soybean lecithin, and sorbitan monooleate derivatives.

Examples of antioxidants are butylated hydroxy anisole (BHA), ascorbic acid and derivatives thereof, tocopherol and derivatives thereof, butylated hydroxy anisole, and cysteine.

Examples of preservatives are parabens, such as methyl or propyl p-hydroxybenzoate, and benzalkonium chloride.

Examples of humectants are glycerin, propylene glycol, sorbitol, and urea.

Examples of penetration enhancers are propylene glycol, DMSO, triethanolamine, N,N-dimethylacetamide, N,N-dimethylformamide, 2-pyrrolidone and derivatives thereof, tetrahydrofurfuryl alcohol, and Azone®.

Examples of chelating agents are sodium EDTA, citric acid, and phosphoric acid.

Examples of other excipients are edible oils like almond oil, castor oil, cacao butter, coconut oil, corn oil, cottonseed oil, linseed oil, olive oil, palm oil, peanut oil, poppyseed oil, rapeseed oil, sesame oil, soybean oil, sunflower oil, and teaseed oil; and of polymers such as carmelose, sodium carmelose, hydroxypropylmethylcellulose, hydroxyethylcellylose, hydroxypropylcellulose, chitosane, pectin, xanthan gum, carragenan, locust bean gum, acacia gum, gelatin, and alginates, Examples of ointment bases are beeswax, paraffin, cetyl palmitate, vegetable oils, sorbitan esters of fatty acids (Span), polyethylene glycols, and condensation products between sorbitan esters of fatty acids and ethylene oxide, e.g′. polyoxyethylene sorbitan monooleate (Tween).

The formulations mentioned above for topical administration may also be applied to wounds or they may be suitable for direct application or for introduction into relevant orifice (s) of the body, e.g. the rectal, urethral, vaginal or oral orifices. The formulation may simply be applied directly on the part to be treated such as, e.g., the mucosa.

### Parenteral Formulations

The pharmaceutical composition may also be administered parenterally by injection, infusion or implantation (intravenous, intramuscular, intraarticular, subcutaneous or the like) in dosage forms, formulations or e.g. suitable delivery devices or implants containing conventional, non-toxic pharmaceutically acceptable carriers and adjuvants.

The formulation and preparation of such compositions is well-known to those skilled in the art of pharmaceutical formulation. Specific formulations can be found in the textbook entitled “Remington's Pharmaceutical Sciences”.

Compositions for parenteral use may be presented in unit dosage forms, e.g. in ampoules, or in vials containing several doses and in which a suitable preservative may be added (see below). The composition may be in form of a solution, a suspension, an emulsion, an infusion device or a delivery device for implantation or it may be presented as a dry powder to be reconstituted with water or another suitable vehicle before use. Apart from the FIBCD1 binding molecules and/or the non-FIBCD1 binding molecules described herein, the compositions may comprise suitable parenterally acceptable carriers and/or excipients or the active drug substance may be incorporated into microspheres, microcapsules, nanoparticles, liposomes or the like for controlled release. Furthermore, the composition may, in addition, conveniently comprise suspending, solubilising, stabilising, pH-adjusting agents and/or dispersing agents.

In another interesting embodiment of the invention, the pharmaceutical composition is a solid dosage form, such as a tablet, prepared from the particulate material described in WO 03/004001 and WO 2004/062643.

As indicated above, the pharmaceutical compositions may contain the FIBCD1 binding molecules and/or the non-FIBCD1 binding molecules in the form of a sterile injection. To prepare such a composition, the FIBCD1 binding molecules and/or the non-FIBCD1 binding molecules is dissolved or suspended in a parenterally acceptable liquid vehicle.

Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives, for example, methyl, ethyl or n-propyl p-hydroxybenzoate. In cases where FIBCD1 binding molecules and/or the non-FIBCD1 binding molecules are only sparingly or slightly soluble in water, a dissolution enhancing or solubilising agent can be added or the solvent may apart from water comprise 10-60% w/w of propylene glycol or the like.

### Dosages

As discussed in detail previously, an important aspect of the present invention is the realisation that the FIBCD1 binding molecules and/or the non-FIBCD1 binding molecules described herein are capable of treating, preventing or curing allergies when administered in clinical relevant amounts, i.e. in amounts sufficiently small to avoid the severe side effects normally associated with the FIBCD1 binding molecules and/or the non-FIBCD1 binding molecules described herein.

It will be understood that the dosage to be administered will be dependent on the administration form (as described herein). Independently, of the administration form, the FIBCD1 binding molecules and/or the non-FIBCD1 binding molecules should be administered in clinically relevant amounts, i.e. in amounts which on the one hand exert the relevant therapeutic effect, but on the other hand does not provide severe side effects.

The invention is further illustrated by the below, non-limiting, examples.

### EXAMPLES

### Buffers

The buffers which are described in this section were used in the procedures described below.

Tris-buffered saline (TBS): 140 mM NaCl, 10 mM Tris-HCl, 0.02% (w/v) NaN₃, pH 7.4; TBS/Tw: TBS containing 0.05% (v/v) Tween 20 (polyoxyethylene sorbitan monolaurate, MERCK-Schuchardt, Hohenbrunn, Germany); phosphate buffered saline (PBS): 137 mM NaCl, 3 mM KCl, 8 mM Na₂HPO₄, 1.5 mM KH₂PO₄, pH 7.4; coating buffer: 60 mM Na₂CO₃, 35 mM NaHCO₃, 0.02% (w/v) NaN₃, pH 9.6; substrate buffer: 100 mM Tris-HCl, 5 mM MgCl₂, 100 mM NaCl, pH 9.5.

### Cloning of Full-Length FIBCD1 and the Ectodomain of FIBCD1

The various FIBCD1 constructs were generated by PCR using Pwo polymerase (Roche, Manheim, Germany) and employing as template I.M.A.G.E clone I.D 4811679 (GenBank™ accession number BCO32953). Full-length V5-His tagged FIBCD1 generated with the primers: 5′GTCCTGCCGGAAGATGGT-3′ and 5′GCGGTCCTCCCGGACCGG-3′; Full-length FIBCD1 generated with the primers: 5′GTCCTGCCGGAAGATGGT-3′ 5′GTCTAGCGGTCCTCCCGGACC-3′. The V5-His tagged putative ectodomain of FIBCD1, encoding amino acid 54-461, was generated by PCR with the primers 5′-CTGAACCACGCCCACGCGCC-3′ and 5″-GCGGTCCTCCCGGACCGGCC-3′; FIBCD1 ectodomain was generated with the primers: 5′CTGAACCACGCCCACGCGCC-3′ and 5′GTCTAGCGGTCCTCCCGGACC-3′. The PCR products were cloned into the expression vectors pSecTag/FRT/V5-His TOPO® TA (FIBCD1 ectodomain) or pcDNA/FRT/V5-His TOPO® TA (FIBCD1 full-length) both vectors from Invitrogen, Carlsbad, Calif. using stand molecular biology techniques. The inserts were sequenced in their entirety.

Generation of HEK293 and CHO cells expressing recombinant forms of FIBCD1 In order to generate cells expressing recombinant forms of FIBCD1, the Flp-In™ system (Invitrogen) was utilized. In this system the cDNA is integrated into a specific genomic site. Flp-In™ T-REx™ HEK293 cells or CHO Flp-In™ T-REx™ (Invitrogen) was used as host cells. HEK293 cells were cultured in Dulbecco's modified Eagle medium (DMEM; Invitrogen) and CHO cells in Hams F12 medium (Invitrogen) both containing penicillin (100 units/ml), streptomycin (100 μg/ml), 2 mM L-glutamine and 10% fetal bovine serum (Invitrogen). At approximately 50% confluency, cells were co-transfected (ratio 1:9) with the pSecTag/FRT/V5-His TOPO® TA or pcDNA/FRT/V5-His TOPO® TA containing the FIBCD1 construct and the pOG44 vector encoding Flp recombinase, using the jetPEI™ transfection kit (Polyplus-transfection SAS, Illkirch cedex, France). The recombinase mediates insertion of the expression vectors into the genomic integration site. Integration of the vectors confers hygromycin resistance. Thus, 48 hours after transfection the HEK293 and CHO cells were selected for stable integration of the FIBCD1 constructs with 150 μg/ml and 800 μg/ml hygromycin B (Invitrogen), respectively. Hygromycin resistant clones were obtained after two weeks of culture. At confluence cells were transferred to DMEM or Hams F12 medium without serum. Supernatants were harvested after 72 hours of expression and analyzed by SDS-PAGE and Western blotting. Mutations was performed using site-directed mutagenesis according to the manufactured recommendations (Stratagene, QuikChange Site-Directed Mutagenesis Kit).

### Purification of FIBCD1-V5 Ectodomain

The above supernatant was dialysed into PBS (0.5 M NaCl), and applied to a HiTrap Chelating HP (Phamacia) column on a FPLC system (Amersham Biosciences, Freiburg, Germany). Proteins bound through the His-tag were eluted by applying an imidazole gradient to the column.

### Chemical Cross-Linking of FIBCD1-V5-his Ectodomain

The FIBCD1-V5-His ectodomain was cross-linked using bis(sulphosuccinimidyl) suberate (BS3). Briefly, BS3 was added to the recombinant FIBCD1-V5-His ectodomain fractions in a 10- to 320 moleclar excess, incubated at room temperature for 30 min, and the reactions stopped by the addition of 0.1 M Tris/HCl buffer, pH 7.4. The cross-linked samples were reduced and analysed by SDS-PAGE and Western blotting.

### Alexa488 Labelling of Acetylated BSA

Alexa488 labelled acetylated BSA was prepared in PBS pH 7.4 and the thiols for coupling were generated by reducing cystine disulfides with the reducing agent tris-(2-carboxyethyl)phosphine (TCEP, Sigma) at a concentration of 10 mM. At a concentration of 2.5 mg/ml acetylated BSA was incubated with Alexa Fluor 488 C5 maleimide (Invitrogen) in a 1:10 molar ratio for 2 hours at room temperature followed by extensive dialysis against PBS to remove uncoupled dye.

### Endocytosis of ¹²⁵I Acetylated BSA

Endocytosis experiments were performed with FIBCD1 expressing CHO cells or non-transfected Flp-Inä CHO cells grown to confluence in 24-well plates essentially as described (Fyfe J C et al 2004 Blood 103:1573-9). In brief, acetylated BSA was labelled with ¹²⁵I using the chloroamine-T method, and triplicates of cells were incubated in Hams F12 medium supplemented with 1% BSA containing 4000 cpm of ¹²⁵I labelled acetylated BSA for various time intervals at 37° C.

SDS-PAGE and Western blotting

SDS-PAGE samples were reduced by heating at 100° C. for 1 min in sample buffer containing 60 mM dithiothreitol and following alkylated by the addition of iodoacetamide to a final concentration of 90 mM. Unreduced samples were heated at 100° C. for 1 min in sample buffer and alkylated by the addition of iodoacetamide to a final concentration of 90 mM. Proteins were separated on 4-12% polyacrylamide gradient gels in a discontinous buffer system and blotted onto polyvinylidene difluoride membrane (Immobilon P, Millipore, Bedford, Mass., USA). The membrane was incubated with 0.5 μg/ml monoclonal mouse anti-V5 antibody or 0.5 μg/ml HG-HYB-12-2 followed by alkaline phosphatase-coupled rabbit anti-mouse IgG (Sigma-Aldrich) diluted 1:1000 in TBS/Tw (500 mM NaCl, 10 mM Tris-HCl, 0.02% (w/v) NaN₃, 0.05% (v/v, pH 7.4) Tween 20). The membrane was washed and developed with 0.1 mg/ml nitro blue tetrazolium (N-6876, Sigma-Aldrich) and 0.17 mg/ml potassium 5-bromo-4-chloro-3-indolylphosphate (B-6149, Sigma-Aldrich) in substrate buffer (100 mM Tris-HCl, 5 mM MgCl₂, 100 mM NaCl, pH 9.5) until sufficient color development was obtained. The markers used were Mark12 (Invitrogen) and Precision Plus Protein Standards (BioRad).

### Deglycosylation of FIBCD1

The presence of N-linked saccharides on FIBCD1 ectodomain that was expressed in HEK-293 cells was demonstrated by enzymatic digestion with PNGaseF (New England Biolabs, Beverly, Mass.), an amidase which cleaves between the innermost N-acetylglycosamine and aspararagine residues of high mannose, hybrid, and complex oligosaccharides from N-linked glycoproteins, for 1 h at 37° C.

### Purification of the Non-Tagged FIBCD1 Ectodomain on an N-Acetylated Immobilized Resin

Toyopearl AF-Amino-650M resin (Tosoh, Tokyo, Japan) (5 ml) was washed twice with distilled water and mixed with 4 ml of 0.2 M sodium acetate and 2 ml of acetic anhydride and then incubated on ice for 30 min. After incubation, 2 ml of acetic anhydride was added to the mixture, and the incubation continued for further 30 min as described by (Gokudan, S. et al., Proc. Natl. Acad. Sci. USA 96; 10086 (1999). The resin was washed several times with distilled water and 1 M NaOH followed by washing with TBS (0.5 M NaCl and 5 mM CaCl₂) before chromatography. Culture supernatant from HEK293 cells was applied to the resin and washed extensively with TBS (0.5 M NaCl and 5 mM CaCl₂). Bound proteins were eluted in TBS (250 mM NaAcetate).

### Characterization of FIBCD1 Acetyl Group Binding

An ELISA system was used to evaluate the ability of various acetylated and nonacetylated compounds to inhibit the binding between FIBCD1 ectodomain and acetylated BSA (Sigma). Microtiter plates (NUNC Maxisorp, Denmark) were coated with acetylated BSA (Sigma) or BSA (Sigma) and blocked with TBS/Tw before being incubated with FIBCD1 ectodomain samples diluted in TBS/Tw (5 mM CaCl₂). The binding between FIBCD1 and acetylated BSA was also tested in the presence of 10 mM EDTA, 5 mM MnCl₂ or 5 mM MgCl₂ instead of CaCl₂. After incubation overnight at 4° C. and washing, the wells were incubated for 2 h at room temperature with chicken anti-FIBCD1 antibodies diluted 1:1000 in TBS/Tw (5 mM CaCl₂). The plates were washed with TBS/Tw (5 mM CaCl₂) and incubated for 1 h with alcalic phosphatase-labelled anti-chicken IgY (whole molecule) (Sigma-Aldrich) diluted 1:1500 in TBS/Tw (5 mM CaCl₂) followed by washing and developed with 1 mg/ml para-nitrophenylphosphate, disodium salt (PNPP) (Boehringer Mannheim, Germany) in substrate buffer.

The specificity of the binding to acetylated BSA was assayed by inhibition with acetylated and non acetylated compounds including glucose (Glc), alpha-methylglucose (alpha-methylGlc), glucosamine (GlcN), N-acetyl glucosamine (GlcNAc), a mannose (Man), alpha-methylmannosamine mannosamine (ManN), N-acetyl mannosamine (ManNAc), galactose (Gal), galactosamine (GalN), N-acetylgalactoseamine (GalNAc), acetyl cholin, L-alanine, N-acetylalanine, sodium acetate, sodium propionate, sodium butyrate and N-acetylneuraminic acid (sialic acid) were all from Sigma-Aldrich. Samples of recombinant FIBCD1 ectodomain (100 ng/ml) were mixed with inhibitors to give a final inhibitor concentration of 50, 25, 12.5, 6.25, 3.13, 1.56, 0.78 and 0.39 mM in TBS/Tw (5 mM CaCl₂). The mixtures were incubated overnight in the acetylated BSA coated wells overnight at 4° C. and processed as described above.

### Production of Chicken Anti-FIBCD1 Antibodies

The immunizations and the purification were done by Davids biotechnology, Regensburg, Germany. Briefly, 1 hen (White Leghorn) was immunized 3 times with approximately 30 μg of FIBCD1-V5-His ectodomain with 2 weeks interval. The collection of the first eggs started 43 days after the first immunization. The purification of IgY polyclonal antibodies was performed using a stepwise salting out method.

### Production of Anti-FIBCD1 Monoclonal Antibodies

Balb/c mice were immunized for the production of monoclonal antibodies against FIBCD1 ectodomain. Mice were immunized with approximately 5 μg recombinant FIBCD1 ectodomain. The mice were immunized 5 times with 2 weeks between each immunization. For the first immunization, the antigen solution was emulsified with 1 volume of Freund's complete adjuvant (Statens Serum Institute, Copenhagen, Denmark). Freunds incomplete adjuvant (Statens Serum Institute) was used for the next 4 immunizations. The mouse was boosted 3 times with 10 μg FIBCD1 ectodomain protein diluted in 200 μl of PBS by injection into the dorsal tail vein. B-cell hybridomas were produced by fusion between myeloma cells (ATCC, CRL-2016, Sp2/mll-6) and spleen cells obtained from the FIBCD1 immunized mice, using polyethylene glycol 1500 as the fusogen as described previously (Köler G, Milstein C. (1975) Nature. 256:495-7). Anti-FIBCD1 ectodomain clones were identified by a direct ELISA using MaxiSorp plates (Nalge-Nunc International, Kamstrup, Denmark) coated with FIBCD1 ectodomain protein diluted in coating buffer (1 μg/ml). Cells producing anti-FIBCD1 antibodies were selected and grown again and the procedure was repeated until a high reactivity from all wells with grown cells was obtained.

### Antibody Purification

The FPLC apparatus (Amersham Pharmacia Biotech) was programmed to standard Protein G purification. The column was washed in PBS with 0.5 M NaCl. The culture supernatant was applied to the column and antibodies eluted by means of citric acid. Antibody containing fractions were neutralized immediately with Na₂CO₃ and pooled. The pool of purified antibody was dialyzed against TBS and diluted to 1 mg/ml.

### Immunohistochemistry

Normal human tissues were obtained from the tissue bank at the Department of Pathology, Odense University Hospital (Odense, Denmark). The tissues were fixed in 4% formalin in PBS for 24 hours and then conventionally dehydrated and embedded in paraffin. A biotin-streptavidin immunoperoxidase technique was used on paraffin sections. Paraffin sections were pre-treated in TEG buffer (10 mmol/L Tris, 0.5 mmol/L. EGTA, pH 9), in a microwave oven for three 5 min periods at 650 W. The sections were left in TEG buffer for 15 min, washed in TBS, pre-incubated with 2% (w/v) BSA in TBS for 10 min, and incubated for 30 min with the mouse anti-human FIBCD1 (Hyb-12-2, 0.5 μg/ml) in TBS containing 15% (w/v) BSA. Slides were washed with TBS prior to incubation for 30 min with biotin-labelled goat anti-mouse Ig (DakoCytomation, Glostrup, Denmark) diluted 1:200 (5 μg/ml) in TBS. After washing with TBS, samples were incubated with HRP-coupled streptavidin (DakoCytomation) diluted in TBS without azide, washed with TBS and water, incubated for 20 min. with 0.04% 3-amino-9-ethylcarbazole (Sigma-Aldrich) in 0.015% H₂O₂, 50 mM sodium acetate buffer, pH 5.0, counterstained with Mayer's hematoxylin for 2 min, and mounted in Aquatex (Sigma-Aldrich). The specificity of the immuno-staining was verified by replacing the primary antibody with a non-specific antibody. The local ethical committee in Odense approved the use of the tissue sections samples (ref. no:

### VF20050070).

### Quantitative RT-PCR

Oligonucleotide primers and probes were obtained from Applied Biosystems, Inc., Foster City, Calif. to monitor the real-time expression of FIBCD1 and beta-actin. Real-time quantitative PCR was performed with the ABI Prisms 7500 (Applied Biosystem) sequence detection system, using Tagman reagents, according to the manufacturer's instructions. The FIBCD1 threshold cycle (Cₜ) was determined and the Cₜ was normalized using the Cₜ of beta-actin to obtain ΔCₜ. To compare relative levels of gene expression of FIBCD1 in different tissues, ΔΔCₜ were calculated using the lowest level of expression as base, which were then converted to real fold expression difference values.

### FACS Analysis

CHO cells expressing full-length FIBCD1-V5-His were incubated with 10 μg/ml anti-V5 or 10 μg/ml anti-FIBCD1 (Hyb-12-1) antibodies in FACS buffer (PBS, 1% BSA and 0.05% azide) for 30 min at 4° C. and washed two times in FACS buffer and incubated with fluorescein isothiocyanate (FITC)-conjugated F(ab′)2 goat anti-mouse (DakoCy-tomation, Glostrup, Denmark) for 30 min and washed three times with FACS buffer.

### Sequence Analysis

DNA sequence analysis, alignments, and amino acid sequence prediction were done by DNAStar's Lasergene software (Madison, Wis., USA). Multiple sequence alignments were conducted using ClustalW algorithm (http://www.ebi.ac.uk/clustalw). Membrane topology and putative membrane-spanning domains were determined by Hidden Markov Model analysis software (http://www.cbs.dtu.dk/services/TMHMM/).

Abbreviations used herein:

| | |
|---|---|
| BSA | bovine serum albumin |
| cDNA | complementary DNA |
| ELISA | enzyme linked immune |
| FIBCD1 | fibrinogen C domain containing 1 (herein it refers |
| | protein, mRNA and cDNA) |
| FIBCD1FReD | fibrinogen C domain containing 1 fibrinogen-related |
| | domain |
| FPLC | fast protein liquid chromatography |
| HEK294 | human embryonic kidney 293 cells |
| LNA | locked nucleic acid |
| LPS | lipopolysaccharide |
| LTA | lipoteichoid acid |
| NAc-BSA | N-acetyl bovine serum albumin |
| NAcMan | N-acetyl-mannosamine |
| PAMP | pathogen associated molecular pattern |
| PBS | phosphate buffered saline |
| PNA | peptide nucleic acid |
| RT-PCR | real time polymerase chain reaction |
| SDS-Page | sodium dodecyl sulfate polyacrylamide gel electrophoresis |
| TBS | Tris-buffered saline |
| TBS/Tw | TBS containing 0.05% (v/v) Tween 20 (polyoxyethylene |
| | sorbitan monolaurate) |
| Th2 | T-helper 2 cells |

1. A pharmaceutical composition comprising:
one or more FIBCD 1 binding molecules or pharmaceutically acceptable salts, esters, and amides thereof; and
optionally one or more non-FIBCD1 binding molecules or pharmaceutically acceptable salts, esters, and amides thereof;

2-40. (canceled)

41. A method for preventing or treating a disease selected from the group consisting of an allergic disease and an inflammatory bowel disease comprising administering to a subject an effective dose of a pharmaceutical composition comprising one or more FIBCD1 binding molecules or pharmaceutically acceptable salts, esters, and amides thereof; and optionally one or more non-FIBCD1 binding molecules or pharmaceutically acceptable salts, esters, and amides thereof, thereby treating or preventing said disease.

42. The method for preventing or treating the disease according to claim 41, wherein the allergic disease is selected from the group comprising: allergic asthma, asthma, extrinsic bronchial asthma, chronic obstructive pulmonary disease, hay fever (seasonal allergic rhinitis), allergic rhinitis, allergic conjunctivitis, hives, eczema, urticaria, angioedema, onchocercal dermatitis, atopic dermatitis, dermatitis, swelling, hypersensitivity pneumonitis, bronchopulmonary dysplasia, food allergy, perfume allergy, drug allergy, and/or other allergic diseases.

43. The method for preventing or treating the disease according to claim 41, wherein the inflammatory bowel disease is selected from the group comprising: Crohn's disease, ulcerative colitis, collagenous colitis, lymphocytic colitis, ischaemic colitis, diversion colitis, Behcet's syndrome, infective colitis, indeterminate colitis, and/or other inflammatory bowel diseases.

44. The method for preventing or treating the disease according to claim 41, wherein the one or more FIBCD 1 binding molecule contains one or more acetyl groups, acetate or is an acetyl radical.

45. The method for preventing or treating the disease according to claim 41, wherein the one or more FIBCD1 binding molecules is selected from the group comprising; monomers, dimers, trimers, tetramers, pentamers, hexamers, longer oligomers and polymers and, where possible, partially deacetylated analogous of D- and L-sialic acid, N-acetyl-D- and L-glucosamine, N-acetyl-D- and L-mannosamine, N-acetyl-D- and L-galactosamine, N-acetylalanine, acetylated D- and L-carbohydrates, N-acetylated D- and L-carbohydrates, acetylated D- and L-amino acids, N-acetylated D- and L-amino acids, acetylated natural nucleic acids, acetylated non-natural nucleic acids, acetylcholine, chitin, or mixtures thereof and pharmaceutically acceptable salts, esters, and amides thereof.

46. The method for preventing or treating the disease according to claim 41, wherein the one or more FIBCD1 binding molecules is selected from the group comprising: chitin, partially deacetylated chitin, monomers, dimers, trimers, tetramers, pen-tamers, hexamers and longer oligomers, polymers and, where possible, partially deacetylated analogous of D- and L-sialic acid, N-acetyl-D- and L-glucosamine, N-62 acetyl-D- and L-mannosamine, or mixtures thereof and pharmaceutically acceptable salts, esters, and amides thereof.

47. The method for preventing or treating the disease according to claim 41, wherein the one or more non-FIBCD1 binding molecules is a drug selected from the group comprising anti-asthma drugs, anti-allergy drugs, anti-histamine drugs, smooth muscle cell relaxing drugs, mast-cell stabilizers, anti-IgE drugs, selective or nonselective potassium channel activators (bronchodilators), immunomodulating agents, mucus secretion inhibitors, mucus liquefying agents, leukotriens modifier drugs, leukot-riens receptor antagonists, mesalamine, steroids, prednisone, Asacol, and Immunosuppressants such as prednisone, infliximab (Remicade), azathioprine (Imuran), methotrexate, or 6-mercaptopurine.

48. The method for preventing or treating the disease according to claim 41, wherein the subject is diagnosed as having an increased risk for the development of allergic diseases.

49. The method for preventing or treating the disease according to claim 41, wherein the subject is diagnosed as having an increased risk for the development of inflammatory bowel disease.

50. The method for preventing or treating the disease according to claim 41, wherein the one or more FIBCD1 binding molecules is selected from the group consisting of one or more antibodies that inhibits or stimulates the function of the naturally occurring FIBCD1 and one or more antisense oligonucleotides that binds to the naturally occurring FIBCD1 and inhibits the transcription and/or the translation of FIBCD1.
